SEQUENCE LISTING (29 sequences)

SEQ ID NO: 1 - DNA, 489 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - DNA, 1610 bp, Homo sapiens
cccagagcagcgctcgccacctccccccggcctgggcagcgctcgcccggggagtccagcggtgtcctgtggagctgccgccatggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaaactcggggaaaccagcccagctaagtccggagtgaaggagcctctctgctttagctaaagacgactgagaagaggtgcaaggaagcgggctccaggagcaagctcaccaggcctctcagaagtcccagcaggatctcacggactgccgggtcggcgcctcctgcgcgagggagcaggttctccgcattcccatgggcaccacctgcctgcctgtcgtgccttggacccagggcccagcttcccaggagagaccaaaggcttctgagcaggatttttatttcattacagtgtgagctgcctggaatacatgtggtaatgaaataaaaaccctgccccgaatcttccgtccctcatcctaacttgcagttcacagagaaaagtgacatacccaaagctctctgtcaattacaaggcttctcctggcgtgggagacgtctacagggaagacaccagcgtttgggcttctaaccaccctgtctccagctgctctgcacacatggacagggacctgggaaaggtgggagagatgctgagcccagcgaatcctctccattgaaggattcaggaagaagaaaactcaactcagtgccattttacgaatatatgcgtttatatttatacttccttgtctattatatctatacattatatattatttgtattttgacattgtaccttgtataaacaaaataaaacatctattttcaatatttttaaaatgca

SEQ ID NO: 5 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 6 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 7 - DNA, 74 bp, Artificial sequence
  Synthetic oligonucleotide Kozak and signal sequence
gatccatcagagcatggctgtactagtactattcctgtgcttagtagctttcccatcatgtgtactaagcgctg

SEQ ID NO: 8 - DNA, 74 bp, Artificial sequence
  Synthetic oligonucleotide Kozak and signal sequence
aattcagcgcttagtacacatgatgggaaagctactaagcacaggaatagtactagtacagccatgctctgatg

SEQ ID NO: 9 - DNA, 24 bp, Artificial sequence
  Synthetic oligonucleotide primer
gatatcacgtgccctccccccatg

SEQ ID NO: 10 - DNA, 53 bp, Artificial sequence
  Synthetic oligonucleotide primer
gatatccaccttggtgttgctgggcttgtgagtggtgtcgctgtggccctgtg

SEQ ID NO: 11 - DNA, 26 bp, Artificial sequence
  Synthetic oligonucleotide primer
tttaaaactcacacatgcccaccgtg

SEQ ID NO: 12 - DNA, 23 bp, Artificial sequence
  Synthetic oligonucleotide primer
ctggcactcatttacccggagac

SEQ ID NO: 13 - DNA, 2078 bp, Artificial sequence
  Nucleic acid encoding an IL-15R IgFc fusion polypeptide
tcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccatcagagcatggctgtactagtactattcctgtgcttagtagctttcccatcatgtgtactaagcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactcccaaatcttgtgataaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgccagaagccgaattctgcagatatccatcacactggcggccgctcgagcatgcatctaga

SEQ ID NO: 14 - protein, 426 aa, Artificial sequence
  IL-15R IgFc fusion polypeptide
MAVLVLFLCLVAFPSCVLSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKZ

SEQ ID NO: 15 - protein, 19 aa, Mus musculus
MAVLVLFLCLVAFPSCVLS

SEQ ID NO: 16 - protein, 306 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 17 - protein, 309 aa, Mus musculus
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLDQNVTGNSGINLTCMSKQGHPKPKKMYFLITNSTNEYGDNMQISQDNVTELFSISNSLSLSFPDGVWHMTVVCVLETESMKISSKPLNFTQEFPSAQTYWKEITASVTVALLLVMLLIIVCHKKPNQPSRPSNTASKLERDSNADRETINLKELEPQIASAKPNAE

SEQ ID NO: 18 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 19 - protein, 322 aa, Mus musculus
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKVLVPVLAVLAAAAFVSFIIYRRTRPHRSYTGPKTVQLELTDHA

SEQ ID NO: 20 - protein, 316 aa, Mus musculus
MLRGWGGPSVGVCVRTALGVLCLCLTGAVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGQPLTFPPEALWVTVGLSVCLVVLLVALAFVCWRKIKQSCEEENAGAEDQDGDGEGSKTALRPLKPSENKEDDGQEIA

SEQ ID NO: 21 - protein, 283 aa, Mus musculus
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVSSSAFVAGWALLSLSCCLMLR

SEQ ID NO: 22 - protein, 331 aa, Mus musculus
MEENEYSGYWEPPRKRCCCARRGTQLMLVGLLSTAMWAGLLALLLLWHWETEKNLKQLGDTAIQNVSHVTKDLQKFQSNQLAQKSQVVQMSQNLQELQAEQKQMKAQDSRLSQNLTGLQEDLRNAQSQNSKLSQNLNRLQDDLVNIKSLGLNEKRTASDSLEKLQEEVAKLWIEILISKGTACNICPKNWLHFQQKCYYFGKGSKQWIQARFACSDLQGRLVSIHSQKEQDFLMQHINKKDSWIGLQDLNMEGEFVWSDGSPVGYSNWNPGEPNNGGQGEDCVMMRGSGQWNDAFCRSYLDAWVCEQLATCEISAPLASVTPTRPTPKSEP

SEQ ID NO: 23 - protein, 238 aa, Mus musculus
MSDSKEMGKRQLRPLDEELLTSSHTRHSIKGFGFQTNSGFSSFTGCLVHSQVPLALQVLFLAVCSVLLVVILVKVYKIPSSQEENNQMNVYQELTQLKAGVDRLCRSCPWDWTHFQGSCYFFSVAQKSWNDSATACHNVGAQLVVIKSDEEQNFLQQTSKKRGYTWMGLIDMSKESTWYWVDGSPLTLSFMKYWSKGEPNNLGEEDCAEFRDDGWNDTKCTNKKFWICKKLSTSCPSK

SEQ ID NO: 24 - protein, 237 aa, H Mus musculus
MSDSMESKTQQVVIPEDEECLMSGTRYSDISSRLQTKFGIKSLAEYTKQSRNPLVLQLLSFLFLAGLLLIILILVSKVPSSEVQNKIYQELMQLKAEVHDGLCQPCARDWTFFNGSCYFFSKSQRNWHNSTTACQELGAQLVIIETDEEQTFLQQTSKARGPTWMGLSDMHNEATWHWVDGSPLSPSFTRYWNRGEPNNVGDEDCAEFSGDGWNDLSCDKLLFWICKKVSTSSCTTK

SEQ ID NO: 25 - protein, 208 aa, Mus musculus
MRAPQMGSLGFLDKGHIPLVLQLLFLILFTGLLVAIIIQVSKMPSSEEIQWEHTKQEKMYKDLSQLKSEVDRLCRLCPWDWTFFNGNCYFFSKSQRDWHDSMTACKEMGAQLVIIKSHEEQSFLQQTSKKNSYTWMGLSDLNKEGEWYWLDGSPLSDSFEKYWKKGQPNNVGGQDCVEFRDNGWNDAKCEQRKFWICKKIATTCLSKW

SEQ ID NO: 26 - protein, 219 aa, Mus musculus
MWLEESQMKSKGTRHPQLIPCVFAVVSISFLSACFISTCLVTHHYFLRWTRGSVVKLSDYHTRVTCIREGPQPGATGGTWTCCPVSWRAFQSNCYFPLNDNQTWHESERNCSGMSSHLVTINTEAEQNFVTQLLDKRFSYFLGLADENVEGQWQWVDKTPFNPHTVFWEKGESNDFMEEDCVVLVHVHEKWVWNDFPCHFEVRRICKLPGITFNWKPSK

SEQ ID NO: 27 - protein, 214 aa, Mus musculus
MNSTKSPASHHTERGCFKNSQVLSWTIAGASILFLSGCFITRCVVTYRSSQISGQNLQPHRNIKELSCYSEASGSVKNCCPLNWKHYQSSCYFFSTTTLTWSSSLKNCSDMGAHLVVIDTQEEQEFLFRTKPKRKEFYIGLTDQVVEGQWQWVDDTPFTESLSFWDAGEPNNIVLVEDCATIRDSSNSRKNWNDIPCFYSMPWICEMPEISPLD

SEQ ID NO: 28 - protein, 209 aa, Mus musculus
MVQERQSQGKGVCWTLRLWSAAVISMLLLSTCFIASCVVTYQFIMDQPSRRLYELHTYHSSLTCFSEGTMVSEKMWGCCPNHWKSFGSSCYLISTKENFWSTSEQNCVQMGAHLVVINTEAEQNFITQQLNESLSYFLGLSDPQGNGKWQWIDDTPFSQNVRFWHPHEPNLPEERCVSIVYWNPSKWGWNDVFCDSKHNSICEMKKIYL

SEQ ID NO: 29 - protein, 176 aa, Mus musculus
MVQERQLQGKAVSWSLRLWSAAVISILLLSTCFIASCVDKVWSCCPKDWKLFGSHCYLVPTVFSSASWNKSEENCSRMGAHLVVIHSQEEQDFITGILDIHAAYFIGLWDTGHRQWQWVDQTPYEESVTFWHNGEPSSDNEKCVTVYYRRNIGWGWNDISCNLKQKSVCQMKKINL

(19) United States
(12) Patent Application Publication
(10) Pub. No.: US 2007/0160578 A1
(43) Pub. Date: Jul. 12, 2007

(54) Expansion of natural killer and CD8 T-cells with IL-15R/ligand activator complexes

(75) Inventors: Thomas A. Waldmann, Silver Spring, MD (US); Sigrid Dubois, Washington, DC (US); Juergen Mueller, Washington, DC (US); Meili Zhang, Bethesda, MD (US); Hiral Patel, North Bergen, NJ (US)

Correspondence Address: KLARQUIST SPARKMAN, LLP, 121 S.W. SALMON STREET, SUITE #1600, PORTLAND, OR, 97204-2988, US

(73) Assignee: The Gov. of the USA as represented by the Secretary of the Dep. of Health and Human Services

(21) Appl. No.: 11/639,877

(22) Filed: Dec. 14, 2006

Related U.S. Application Data
(60) Provisional application No. 60/750,639, filed on Dec. 14, 2005.

(51) Int. Cl. A61K 38/20; A61K 39/395

(52) U.S. Cl. 424/85.2; 424/145.1

(57) ABSTRACT

The disclosure provides methods, and compositions for use in methods, for expanding lymphocyte populations in vitro, ex vivo, and in vivo using IL-15Rα/IL-15 activator complexes.

### PRIORITY CLAIM

This claims the benefit of U.S. Provisional Application No. 60/750,639, filed Dec. 14, 2005, which is incorporated herein by reference in its entirety.

### FIELD

This disclosure relates to the field of immunology. More specifically, the disclosure relates to the expansion of lymphocyte populations using IL-15 receptor-ligand complexes.

### BACKGROUND

IL-15 is involved in the generation of the innate immune response through the activation of effector functions of NK cells (Carson et al., J. Clin. Invest. 99:937-943, 1997) and dendritic cells (Ohteki et al., J. Immunol. 166:6509-6513, 2001), and is important in the survival of CD8+ memory T cells (Ku et al., Science 288:675-678, 2000 and Zhang et al., Immunity 8:591-599, 1998) and other aspects of adaptive immunity. However, previous attempts to expand these cell populations in vitro and in vivo with IL-15 have met with only limited success.

IL-15 binds to a specific receptor on T and NK cells. IL-15 and IL-15Rα are co-expressed on activated dendritic cells and on monocytes. IL-15/IL-15α bind as a heterodimer to two chains on T and NK cells, namely IL-15Rβ and γc molecules. The β and γc chains are shared between IL-2 and IL-15 and are essential for the signaling of these cytokines (Giri et al., EMBO J. 13:2822-2830, 1994 and Giri et al., EMBO J. 14:3654-3663, 1995). Consistent with the sharing of IL-2/15βγc receptor complex, numerous studies have shown that IL-15 mediates many functions similar to those of IL-2 in vitro (reviewed in Waldmann and Tagaya, Annu. Rev. Immunol. 17:19-49, 1999). However, IL-15 also makes distinct contributions to the life and the death of T lymphocytes.

The biological effects of IL-15 are mediated via the formation of a membrane-bound complex of IL-15 associated with IL-15Rα on the surface of the cell. IL-15/IL-15Rα on the surface of one cell stimulates in trans neighboring cells. Cell surface IL-15/IL-15Rα is substantially more biologically active than soluble IL-15 alone, and the cell associated IL-15/IL-15Rα complex can efficiently stimulate the proliferation of both βγ- and IL-15Rαβγ-bearing cells at picomolar concentrations (Dubois et al., Immunity 17:537-47, 2002). Because the biological effects of IL-15 have thus far required cellular presentation of the ligand-receptor complex, it has not been possible to fully utilize IL-15 in vitro or in vivo to expand cell populations to enhance specific or innate immune responses. Indeed, administration of a soluble form of the IL-15Rα consisting of the extracellular ligand-binding domain of IL-15Rα, although capable of binding IL-15, was found to act as an antagonist of IL-15 (Ruchatz et al., J Immunol. 160:5654-60, 1998; Smith et al., J Immunol. 165:3444-50, 2000).

The present disclosure overcomes these problems, and provides methods for expanding populations of lymphocytes, and enhancing a variety of immune functions.

### SUMMARY

The present disclosure concerns methods for expanding populations of lymphocytes using molecular complexes that include a polypeptide including the extracellular ligand-binding domain of the IL-15Rα and an IL-15Rα ligand, such as an IL-15 polypeptide. Methods for expanding lymphocytes, and particular subsets thereof, involve contacting cells with IL-15Rα/ligand activator complexes in vitro, ex vivo or in vivo. Methods are also disclosed for treating subjects with cancer and for enhancing immune responses, such as the immune response against a pathogen or a vaccine, using IL-15Rα/ligand activator complexes.

The foregoing and other features and advantages of the invention will become more apparent from the following detailed description, which proceeds with reference to the accompanying figures.

### BRIEF DESCRIPTION OF THE DRAWINGS

FIG. 1 is a schematic illustration of an exemplary IL-15Rα/IL-15 activator complex.

FIGS. 2A-C are a set of cytometry-generated dot plots illustrating expansion of murine NK (NK1.1⁺), CD8 Memory Phenotype (CD8⁺) and CD8 Natural Killer T (NK1.1⁺/CD8⁺) cells in cultures blood (A), spleen (B) and bone marrow (C) cultured for seven days with 1 nM murine IL-15RαIgFc/IL-15 complex.

FIG. 3 is a set of cytometry-generated histograms and dot blots illustrating expansion of human NK (CD3⁻) CD56⁺) and CD8MP (CD3⁺/CD8⁺) cells in blood cultured for ten days with 1 nM human IL-15RαIgFc/IL-15 complex.

FIG. 4 is a line graph illustrating thymidine incorporation following culture of murine blood derived cells with increasing concentrations of IL-15 or IL-15RαIgFc/IL-15.

FIG. 5 is a line graph showing the proliferative effect of human IL-15RαIgFc/IL-15 complex on human NK cells.

FIG. 6 is a line graph comparing proliferative effect of IL-15, IL-15/IL-15Rα and IL-15RαIgFc/IL-15.

FIG. 7 is a set of bar graphs comparing the proliferative effect of IL-15, IL-15RαIgG1Fc/IL-15, IL-15α/IL-15, and IL-15RαIgG4Fc/IL-15, in the presence or absence of cross-linking IgG1. Note the different measures on the y-axes.

FIG. 8 is a line graph illustrating lysis of the syngeneic tumor MC38 by IL-15/IL-15Rα-Fc complex-induced blood NK cells.

FIG. 9 is a set of histograms showing expansion of NK cells (top panel) and CD8⁺ memory phenotype T (CD8MP) cells (bottom panel) following administration of IL-15 or IL-15RαIgFc/IL-15 complexes to mice. Samples were obtained seven days after treatment, and evaluated by flow cytometry.

FIG. 10 is a set of cytometry-generated dot blots comparing the expansion of NK cells (NK1.1⁺) by L-15RαIgG1Fc/IL-15 in wild-type and mice deficient for the Fc receptor chain FcRγ.

FIGS. 11A-11D are a set of graphs showing the lysis activity of sIL-15 complex-expanded NK cells. ⁵¹Cr-labeled target cells were co-incubated with NK cells for 4 h at various effector:target ratios. Lysis activity was assessed by the amount of radioactivity in the supernatant. Values shown are averages ±SD from three experiments. A, NK cells lyse MC38 and Yac-1 but not EL4 cells. B, A 24-h pretreatment of B16 melanoma cells with IFN-γ inhibited NK cell-mediated lysis. The insert depicts the levels of MHC I that were expressed by the same B16 cells. C, sIL-15 complex-cultured NK cells that were derived from IL-15^{-/-} mice showed similar lysis activity towards MC38 when compared with wild-type cells. D, Freshly isolated NK cells were tested for their ability to lyse MC38. Prior injections of sIL-15 complex (10 μg 7 and 4 days before isolation) increased their lysis activity. However, levels stayed below those of cultured NK cells (compare with A).

FIG. 12 is a graph showing the survival increase of B16-bearing mice by sIL-15 complex administration. Three groups often mice each were injected with 10⁶ B16 melanoma cells. Mice were treated with PBS or with 9 doses of 2 μg IL-15 or 10 μg sIL-15 complex. Treatments with sIL-15 complex increased the survival that was significantly longer (p<0.05) than treatments with PBS or IL-15.

FIG. 13 is a graph of the results obtained in tumor therapy with IL-15 versus sIL-15 complex in the MC38 tumor model.

FIG. 14 is a set of plots showing the effect of alternative activators within the soluble IL-15 complex on the proliferation of CD8 and NK cells in vitro.

### BRIEF DESCRIPTION OF THE SEQUENCE LISTING

SEQ ID NO:1 represents the polynucleotide sequence of an example of a human IL-15.

SEQ ID NO:2 represents the amino acid sequence of an example of a human IL-15.

SEQ ID NO:3 represents the amino acid sequence of an example of a human IL-15 proteolytic cleavage product.

SEQ ID NO:4 represents the polynucleotide sequence of an example of a human IL-15 receptor alpha (IL-15Rα).

SEQ ID NO:5 represents the amino acid sequence of an example of an extracellular domain of human IL-15Rα.

SEQ ID NO:6 represents the amino acid sequence of the sushi domain of an example of a human IL-15Rα.

SEQ ID NO:7 represents the polynucleotide sequence of a synthetic oligonucleotide comprising a murine IgG heavy chain Kozak and signal sequences (sense).

SEQ ID NO:8 represents the polynucleotide sequence of a synthetic oligonucleotide comprising a murine IgG heavy chain Kozak and signal sequences (antisense).

SEQ ID NO:9 represents the polynucleotide sequence of a synthetic oligonucleotide for the amplification of an example of an extracellular domain of human IL-15Rα (forward).

SEQ ID NO:10 represents the polynucleotide sequence of a synthetic oligonucleotide for the amplification of an example of an extracellular domain of human IL-15Rα (reverse).

SEQ ID NO:11 represents the polynucleotide sequence of a synthetic oligonucleotide for the amplification of a human IgG1 Fc domain (forward).

SEQ ID NO:12 represents the polynucleotide sequence of a synthetic oligonucleotide for the amplification of a human IgG1 Fc domain (reverse).

SEQ ID NO:13 represents the polynucleotide sequence of an exemplary nucleic acid that encodes an IL-15RαIgFc fusion polypeptide.

SEQ ID NO:14 represents the amino acid sequence of an exemplary IL-15RαIgFc fusion polypeptide.

SEQ ID NO: 15 represents the amino acid sequence of a murine Ig leader peptide.

SEQ ID NOs: 16-21 represent the amino acid sequences of proteins that cause lymphocyte activation.

SEQ ID NOs: 22-29 are the amino acid sequences of membrane proteins that do not activate lymphocytes.

### DETAILED DESCRIPTION

Introduction

Both interleukin-15 (IL-15) and interleukin-15 receptor alpha (IL-15Rα) play an important role in the proliferation and survival of lymphocyte populations, including Natural killer cells (NK cells) and CD8-positive T cells, such as CD8-positive memory phenotype T cells (CD8MP) and CD8-positive T cells that express NK receptors (CD8NKT). These cells are of medical interest since they can recognize and destroy both tumor cells and pathogen-infected cells, including cells that are infected by viruses such as HIV.

The present disclosure concerns methods for using an artificial complex that includes a ligand, such as an IL-15 polypeptide or fragment or derivative thereof, bound to an extracellular domain of the IL-15Rα. Lymphocyte and/or lymphocyte progenitors are contacted with complexes including IL-15Rα/ligand subunits to expand populations of lymphocytes, including NK cells, CD8MP and CD8NKT cells. The methods disclosed herein are useful for the expansion of such cell populations in vitro and in vivo, and are useful in a variety of contexts in which the enhancement of an immune response is desired.

Thus, one aspect of the disclosure relates to methods for expanding a population of lymphocytes. Examples of such methods involve contacting one or more lymphocytes or lymphocyte progenitors with an IL-15Rα/IL-15 complex. The complex subunits include (1) a polypeptide with an extracellular ligand-binding domain of an IL-15Rα and (2) a ligand thereof, which in combination with the receptor possesses lymphoproliferative activity, such as an IL-15 polypeptide.

The extracellular ligand-binding domain can be a component of a fusion polypeptide that includes, in addition to the extracellular ligand-binding domain of the IL-15Rα, a polypeptide domain that promotes activation of lymphocytes. Some proteins are known to activate NK cells and can be used in this context. In one example, the activation domain is an immunoglobulin Fc (IgFc) domain. Exemplary activation domains include IgG Fc and NK receptor ligands. Exemplary activation domains include CD80, CD86, B7-H1, B7-H2, B7-H3, and B7-H4 activation domains. In one embodiment, activation domain interacts with a lymphocyte membrane protein and results in changes in morphology, proliferation and/or cytokine production by affected lymphocytes.

Contacting the lymphocyte or lymphocyte progenitor with the IL-15Rα/ligand complex results in the expansion of the lymphocyte population by inducing proliferation and/or enhancing survival of cells, including NK cells, CD8MP cells and CD8NKT cells. Contacting the cells with the IL-15Rα/ligand complex can be effected in vitro, for example, using cells obtained from peripheral blood or bone marrow. Such a population can be a mixed population of cells, such as a mixed population of lymphocytes including more than one of NK cells, CD8MP cells, CD8NKT cells, and/or progenitors thereof. Alternatively, the cells can be purified (or isolated) populations of cells, such as NK cells, NK cell progenitors, CD8MP cells, CD8NKT cells, or CD8+ cell progenitors.

For example, a population of cells, including one or more NK cells or NK cell progenitors can be obtained from a subject, and optionally enriched, prior to contacting the cells with activating IL-15Rα/ligand complexes in vitro. Typically, the cells are suspended in tissue culture medium or physiological saline solution. Similarly, CD8⁺ T cells, such as CD8MP cells, or progenitors thereof, can be obtained and contacted in vitro with the IL-15Rα/ligand complexes. Optionally, the CD8⁺ T cells are purified or enriched. The expanded lymphocyte populations can then be transplanted (introduced or administered) into a subject, for example, to enhance an immune response. Alternatively, activating IL-15Rα/ligand complexes are administered to a subject, typically in a composition including a pharmaceutically acceptable excipient or carrier, to expand such populations of lymphocytes in vivo.

Typically, the IL-15Rα/ligand complex is selected to be compatible with and optimally active in the subject. For example, if the subject is a human subject, a human IL-15 polypeptide and a human IL-15Rα polypeptide can be used to expand the lymphocytes. If the subject is a non-human subject, appropriate ligand and IL-15Rα molecules are selected based on the species of the subject (for example, if the subject is a mouse, complexes including murine IL-15 and IL-15Rα complexes can be used, etc.).

Populations of lymphocytes (including NK cells, CD8MP and/or CD8NKT cells) expanded with the activating IL-15Rα/ligand complexes described herein are useful for enhancing an immune response in a variety of circumstances. For example, NK cell populations expanded as disclosed herein are particularly effective for the treatment of tumors, such as malignant melanoma and renal cell malignancies, among others. CD8MP cells expanded as described herein are particularly useful for the enhancement of pathogen-specific immune responses, such as immune responses against cells infected with viruses, such as HIV.

Lymphocytes expanded with IL-15Rα/ligand activator complexes can induce death of tumor cells. Hence administration of such complexes forms the basis for methods of treating cancer in a subject. For example, NK cells and/or NK cell progenitors can be obtained from bone marrow or peripheral blood of a subject diagnosed with a cancer, such as malignant melanoma or renal cell carcinoma. The whole peripheral blood or bone marrow, or a population derived therefrom that is enriched for NK cells and/or NK cell progenitors, is contacted with an IL-15Rα/ligand activator complex to substantially expand a population of NK cells. The population of cells can be contacted in vitro (or ex vivo) and then introduced back into the subject from whom the cells were obtained. Similarly, NK cells and/or NK cell progenitors can be obtained from a donor for expansion and introduction into a subject with cancer. Alternatively, NK cell lines derived from a subject can be expanded in vitro with activating IL-15Rα/ligand complexes prior to introduction into the subject for the treatment of cancer. In other embodiments, CD8+ T cells, such as CD8MP and/or CD8NKT cells are expanded and introduced into a subject in order to induce killing of tumor cells. In other embodiments, the IL-15Rα/ligand complexes are administered directly to a subject, effectively contacting and expanding the lymphocyte population(s) in vivo.

Thus, the disclosure relates to methods of treating a subject with cancer by administering to such a subject (a) an activator complex including a plurality of subunits, each of which subunits includes a polypeptide with an extracellular ligand-binding domain of IL-15Rα and a ligand thereof that stimulates the desired lymphoproliferative activity; (b) a population of lymphocytes including CD8⁺ T cells, such as CD8MP cells and/or CD8NKT cells that have been expanded ex vivo with such IL-15Rα/ligand complexes; and/or (c) a population of lymphocytes including NK cells that have been expanded ex vivo with such IL-15Rα/ligand complexes. In specific embodiments the complex includes a plurality of subunits, each of which includes a fusion polypeptide with an extracellular IL-15Rα ligand-binding domain and a domain that promotes activation of lymphocytes, and an IL-15 polypeptide. In exemplary embodiments, the fusion polypeptide includes an IgFc domain that promotes activation of NK, CD8MP and CD8NKT cells.

The disclosure also relates to methods of enhancing an immune response against a pathogen (such as a virus, bacterium, fungus or intracellular parasite) by administering to a subject with a pathogen infection (a) an activator complex including a plurality of subunits, each of which subunits includes a polypeptide with an extracellular ligand-binding domain of IL-15Rα and a ligand thereof that is capable of inducing expansion of lymphocyte populations when coupled with the IL-15Rα; (b) a population of lymphocytes including CD8⁺ T cells, such as CD8MP cells and/or CD8NKT cells that have been expanded ex vivo with such IL-15Rα/ligand complexes; and/or (c) a population of lymphocytes including NK cells that have been expanded ex vivo with such IL-15Rα/ligand complexes. As indicated above, specific embodiments include fusion polypeptides that have an IL-15Rα ligand-binding domain and a domain, such as an IgFc domain that promotes lymphocyte activation.

Another aspect of the disclosure relates to methods for enhancing an immune response to a target antigen, such as a vaccine antigen, by administering a vaccine composition including the target antigen to a subject and an activating IL-15Rα/ligand complex. The IL-15Rα/ligand complex can be administered simultaneously with the target antigen, or sequentially in one or more doses.

Another aspect of the disclosure relates to pharmaceutical compositions suitable for use in the methods disclosed herein. For example, the pharmaceutical compositions disclosed herein contain a pharmaceutically effective amount of an IL-15Rα/ligand activator complex and a pharmaceutically acceptable carrier. Commonly, the IL-15Rα/ligand complexes include a polypeptide, such as a fusion polypeptide, including the extracellular ligand-binding domain of an IL-15Rα bound to a ligand of the receptor, such as an IL-15 polypeptide (or variant or fragment thereof). For example, in one embodiment suitable for human therapeutic applications, the activating IL-15/ligand complex includes a fusion polypeptide with an extracellular ligand-binding domain of a human IL-15Rα and a human IgFc (such as IgG1 Fc) domain bound to a human IL-15 polypeptide.

These and other aspects of the invention will be described in detail under the specific subject headings below.

Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854288-7); Kendrew et al. (eds.), The Encyclopedia of Molecular Biology, published by Marston Book Services Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 047-118634-1).

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as a growth factor, are intended to be approximate. Thus, where a concentration is indicated to be at least (for example) 200 pg, it is intended that the concentration be understood to be at least approximately (or “about” or “˜”) 200 pg.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of this disclosure, the following explanations of specific terms are provided:

The “interleukin-15 receptor” consists of three polypeptides, designated α, β and γ. Whereas the β and γ polypeptides are common to the IL-2 and IL-15 receptors (often referred to as IL-2β and γ common chain), the IL-15Rα subunit is unique to the IL-15 receptor. IL-15Rα is disclosed by U.S. Pat. No. 6,548,065, which is incorporated herein in its entirety. The human IL-15Rα polypeptide is represented by GENBANK® Accession No. Q13261, and the nucleotide sequence encoding the IL-15Rα is represented by GENBANK® Accession No. U31628 (SEQ ID NO:4). With respect to the amino acid sequence identified as Accession No. Q13261 amino acids 31-205 constitute the extracellular domain of the IL-15Rα (SEQ ID NO:5). Amino acids 31-95 (SEQ ID NO:6) have been designated the Sushi domain, which is important for ligand-binding activity. Amino acids 206-228 constitute the transmembrane domain. Thus, polypeptides including an extracellular ligand-binding domain include fragments of a full-length IL-15Rα polypeptide encompassing, at a minimum amino acids 31-95, such as amino acids. Examples of a polypeptide including an extracellular binding domain are amino acids 32-95, amino acids 33-95, amino acids 34-95, amino acids 35-95, amino acids 31-96, amino acids, 31-97, amino acids 31-98, amino acids 31-99 or amino acids 31-100 of SEQ ID NO: 5. More commonly, the extracellular ligand-binding domain includes additional amino acids derived from the IL-15Rα extracellular domain, such as the entire IL-15Rα extracellular domain. In exemplary embodiments, the extracellular ligand-binding domain includes amino acids 31-205 of Accession No. Q13261, however fragments encompassing a subsequence thereof including at a minimum the sushi domain of amino acids 31-95 are also contemplated.

An “IL-15Rα ligand” is a ligand that binds to the IL-15Rα to provide the desired biological activity, such as lymphoproliferative activity, including inducing proliferation and promoting survival of a variety of lymphocyte populations, including natural killer (NK) cells and T cells. Experimentally, IL-15 activity is characterized by the ability to stimulate proliferation of CTLL-2 cells (as described in Gillis and Smith, Nature 268:154-156, 1977), and can be detected at a molecular level based on activation of the JAK/STAT signaling pathway. Exemplary IL-15Rα ligands include the cytokine IL-15, as well as variants and fragments thereof. The amino acid sequence of human IL-15 is represented by GENBANK® Accession No. AAA21551, and SEQ ID NO:2. The nucleic acid represented by SEQ ID NO:1 (corresponding to nucleotides 317-805 of GENBANK® Accession No. U14407) is an exemplary nucleic acid encoding human IL-15. The term, IL-15 also encompasses IL-15 of species other than human, such as non-human primates, mouse, rat, pig, horse, cow, dog, etc.

Similarly, fragments of IL-15, such as amino acids 49-162 of SEQ ID NO:2 (that is, SEQ ID NO:3), which has previously been characterized as a mature form of IL-15 derived by proteolytic cleavage of a leader sequence from the polypeptide of SEQ ID NO:2, and other fragments that retain the biological activity of IL-15 are encompassed by the term IL-15. IL-15 analogs, including derivative or variants of IL-15 having one or more substituted amino acid, that exhibit the biological activity of IL-15 are also included within the meaning of the term IL-15 Rα ligand. Exemplary analogs are described in U.S. Pat. No. 5,552,303 and in Bernard et al., J. Biol. Chem. 279:24313-24322, 2004, which are incorporated herein by reference.

Human IL-15 can be obtained according to the procedures described by Grabstein et al., Science, 264:965-968, 1994, and in U.S. Pat. No. 5,552,303, which are incorporated herein by reference. Alternatively, nucleic acids encoding human and other IL-15 polypeptides can be obtained by conventional procedures such as polymerase chain reaction (PCR) based on DNA sequence information provided in SEQ ID NO:1.

As used herein, the term “complex” refers to an association of two or more macromolecular components, such as polypeptides. For example, an “IL-15Rα/ligand complex” refers to a macromolecular complex in which a first component consisting of all or a portion of an IL-15Rα polypeptide is associated with a second component consisting of a ligand for this receptor, such as all or part of an IL-15 polypeptide. More specifically, the present disclosure relates to biologically active, acellular (that is, soluble) IL-15Rα/ligand complexes that include a first component including all or a portion of an IL-15Rα polypeptide associated with a second component including a ligand, such as an IL-15 polypeptide. In the context of this disclosure, the IL-15Rα polypeptide includes at least a portion of the IL-15Rα including the extracellular ligand-binding domain, for example, the entire extracellular domain or a smaller portion including, at a minimum, the sushi domain.

An IL-15Rα/ligand complex can be in any of a variety of forms, so long as the IL-15Rα/ligand subunits are associated in close enough proximity to interact with, and activate the IL-15Rβγ complex. Activation of the IL-15Rβγ complex can be determined, for example, by proliferation of CTLL-2 cells (or other CD8⁺ T cells or NK cells, as described herein), or by activation of the JAK/STAT signaling pathway. For example, the IL-15Rα component can be in the form of a fusion polypeptide. In the context of this disclosure, an activating IL-15Rα/IL-15 complex (an IL-15Rα/IL-15 activator complex) includes a lymphocyte activation domain that activates lymphocytes (for example, NK cells, CD8MP cells, CD8NKT cells, and/or progenitors thereof) by an IL-15 receptor independent mechanism. Thus, an activating IL-15Rα/IL-15 complex can include a fusion polypeptide with a ligand-binding domain of IL-15Rα and a second polypeptide domain, the lymphocyte activation domain, that promotes lymphocyte activation (such as an IgG1 Fc domain), associated with all or a portion of an IL-15 polypeptide.

A “fusion polypeptide” is a polypeptide consisting of at least two amino acid subsequences originating (or derived from) different proteins. Typically, a fusion polypeptide is encoded by a chimeric gene, in which two or more polynucleotide sequences each originating from a different genomic sequence have been joined (for example, by recombinant DNA procedures) to form a contiguous open reading frame. The subsequences can be derived from different genes of the same species, or from the same or different genes of two or more different species. In some instances, the two or more amino acid subsequences include functional domains or regions of a protein.

When referring to a polypeptide or protein, a “domain” is a structurally defined region of the polypeptide.

A “lymphocyte” is a mononuclear white blood cell. Lymphocytes include T cells, B cells, and natural killer (NK) cells. A “B cell” is a type of lymphocyte that expresses antigen specific cell-surface immunoglobulin. Following binding of the cell-surface immunoglobulin to antigen, B cells can differentiate into antibody producing plasma cells. A “T cell” is a thymus-dependent lymphocyte that expresses a highly polymorphic cell-surface T cell receptor and an associated molecular complex designated by the “cluster of differentiation” marker CD3. T cells include, but are not limited to lymphocytes characterized by the presence of cluster of differentiation markers CD4 and CD8. CD4⁺ T lymphocytes, also known as helper T cells, help regulate the immune response, including antibody responses as well as killer T cell responses. CD8⁺ T cells are characterized by the CD8 cell-surface marker, and include CD8 cytotoxic (CD8CT) or “killer” T cells, as well as CD8 memory phenotype (CD8MP) T cells, among others. T cells expressing CD8 interact with peptides presented by MHC Class I molecules, whereas T cells expressing CD4 interact with peptides presented by MHC Class I molecules.

Natural Killer (NK) cells are large granular lymphocytes involved in the innate immune response. Functionally, they exhibit cytolytic activity against a variety of targets via exocytosis of cytoplasmic granules containing a variety of proteins, including perforin, and granzyme proteases. Killing is triggered in a contact-dependent, non-phagocytotic process which does not require prior sensitization to an antigen. Human NK cells are characterized by the presence of the cell-surface markers CD16 and CD56, and the absence of the T cell receptor (CD3). NKT cells or CD8NKT possess characteristics and cell-surface markers of both T cells and NK cells.

A “progenitor” cell is an immature cell capable of dividing and/or undergoing differentiation into one or more mature effector cells. In the context of this disclosure a lymphocyte progenitor includes, for example, pluripotent hematopoietic stem cells capable of giving rise to mature cells of the B cell, T cell and NK lineages. In the B cell lineage (that is, in the developmental pathway that gives rise to mature B cells), progenitor cells also include pro-B cells and pre-B cells characterized by immunoglobulin gene rearrangement and expression. In the T and NK cell lineages, progenitor cells also include bone-marrow derived bipotential T/NK cell progenitors, as well as intrathymic progenitor cells, including double negative (with respect to CD4 and CD8) and double positive thymocytes (T cell lineage) and committed NK cell progenitors.

In the context of the methods of the present disclosure, the term “in vitro” refers to a method in which cells are manipulated outside of the body of a subject (that is, “ex vivo”). Typically, the cells are placed in a receptacle or container suitable for the growth or maintenance (for at least short periods of time) of cells in a growth medium or buffer. In contrast, the term “in vivo” refers to methods that are performed on cells within the body of a subject. A subject can be either a human subject, or a non-human subject, such as a veterinary subject (for example, a non-human primate, a mouse, a cat, a dog, a goat, a pig, a sheep, a cow or a horse).

Il-15 Receptor Complexes

The methods disclosed herein are based on the observation that protein complexes including subunits characterized by (1) a polypeptide including all or part of the extracellular domain of IL-15Rα and an activator domain, associated with (2) all or part of an IL-15 polypeptide, but not IL-15Rα/IL-15complexes without activator, are capable of inducing expansion of a variety of lymphocyte populations. IL-15Rα/ligand complexes including an activator elicit potent biological effects on certain T cell subclasses (including CD8MP and CD8NKT) and NK cells, which cannot be obtained using soluble IL-15 or complexes consisting of a ligand-receptor pair without activator (for example, IL-15 associated with a soluble IL-15Rα extracellular domain).

A feature of biologically active IL-15Rα/ligand complexes is the presentation of complexes (for example, IL-15 polypeptides) in close proximity to an activator. In an embodiment, described in detail in the EXAMPLES section, the extracellular ligand-binding domain of IL-15Rα is a component of a fusion polypeptide. The fusion polypeptide also includes a second domain, from a polypeptide other than IL-15Rα, that induces activation. Thus, the fusion polypeptide can include a “lymphocyte-activating domain.” In an exemplary embodiment, the fusion polypeptide includes an immunoglobulin Fc domain that induces activation of lymphocytes via binding to and stimulating Fc receptors. For example, the IgFc domain can include an entire Fc region (for example, including the hinge, CH2 and CH3 domains), or a part of an Fc region, such as a hinge region and CH2 domain. Most commonly, the Ig domain is selected from a IgG class immunoglobulin molecule. However, other classes of immunoglobulin Fc domains can also be used. In additional exemplary embodiments, the fusion protein includes a domain of CD80, CD86, B7-H1, B7-H2, B7-H3 or B7-H4. Exemplary domains are disclosed in the examples section below.

The IL-15Rα ligand-binding domain and the lymphocyte-activating domain (for example, an IgFc domain) can originate in the same or different species. Typically, the IL-15Rα domain (and the corresponding IL-15 polypeptide) are selected to correspond to the cell on, or the subject in, which biological activity is desired. In one exemplary embodiment of an activating IL-15Rα polypeptide, the entire extracellular ligand-binding domain of murine IL-15Rα is joined in reading frame with the Fc domain of human immunoglobulin (Ig) G1 so that a contiguous polypeptide is produced. In another exemplary embodiment, the extracellular ligand-binding domain of human IL-15Rα is joined in reading frame with the Fc domain of human IgG1 to produce a contiguous fusion polypeptide. Similarly, immunoglobulin Fc domains from other human immunoglobulin serotypes, or from the immunoglobulins of other species (including humanized immunoglobulin domains) can be employed as activation domains in IL-15Rα fusion polypeptides. In additional exemplary embodiments, the extra-cellular ligand-binding domain of human IL-15Rα is joined with a domain of CD80, CD86, B7-H1, B7-H2, B7-H3 or B7-H4. In several embodiments, in order to reduce antigenicity (immunogenicity) of the fusion polypeptide, for in vivo applications, it is generally desirable to select an activation domain from the species into which the fusion polypeptide is to be introduced or administered.

Typically, polypeptides including the extracellular domain of IL-15Rα (including various fusion polypeptides, such as IL-15RαIgFc fusion polypeptides) are produced by expressing a recombinant nucleic acid in a suitable host cell, and optionally, isolating the expressed fusion polypeptide. For example, a polynucleotide sequence encoding all or part of the extracellular domain of IL-15Rα is ligated in the same translational reading frame as a polynucleotide sequence encoding a polypeptide (or peptide) that promotes or facilitates lymphocyte activation by the resulting fusion polypeptide.

The polynucleotide encoding the IL-15Rα polypeptide is operably linked to a transcriptional regulatory sequence including a promoter (and optionally, one or more enhancers), such that the polypeptide is transcribed and then translated. Thus, expression control sequences can include appropriate promoters, enhancers, transcription terminators, a start codon (typically, ATG) in front of (5′ to) the polynucleotide encoding the fusion polypeptide, as well as one or more stop codons to ensure appropriate termination of translation.

Both constitutive and inducible promoters can be used to control expression of polypeptides including the extracellular domain of IL-15Rα (see e.g., Bitter et al., in Methods in Enzymology, Ray and Grossman (eds.) 153:516-544, 1987, ISBN 0-12-182054-8). For example, when cloning in bacterial systems, inducible promoters such as pL of bacteriophage lambda, plac, ptrp, ptac (ptrp-lac hybrid promoter) and the like can be used. For expression in mammalian cell systems, promoters derived from the genome of mammalian cells (for example, metallothionein promoter) or from mammalian viruses (for example, the cytomegalovirus (CMV) immediate early promoter, the retrovirus long terminal repeat; the adenovirus late promoter; the vaccinia virus 7.5K promoter) can be used. Promoters produced by recombinant DNA or synthetic techniques can also be used to provide for transcription of the nucleic acid sequences. Suitable promoters for expression in non-mammalian eukaryotic cells (such as insect cells, yeast cells, or plant cells, among others) are also well-known in the art, and can be used for expression of IL-5Rα polypeptides.

The nucleic acid encoding the IL-15Rα polypeptide is introduced into a host cell in which the nucleic acid can be propagated (replicated) and the encoded polypeptide expressed. The host cell can be prokaryotic or eukaryotic depending on the selection of appropriate transcription (and translation) regulatory control sequences. The term host cell also includes any progeny of the subject host cell. Host cells including a heterologous nucleic acid are produced (for example, transduced, transformed or transfected) by introducing a vector including the polynucleotide sequence encoding the fusion polypeptide into the cell. As described above, the vector can be in the form of a plasmid, a viral particle, a phage, etc. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as HEK 293, SP2/0, COS, CHO, or BHK cells, plant cells, etc. In certain examples detailed below, nucleic acids encoding the IL-15Rα polypeptides are introduced into HEK 293 cells (ATCC CRL-1573) or SP2/0 cells (ATCC CRL-1581) to produce recombinant IL-15Rα polypeptides (e.g., fusion polypeptides including the extracellular domain of IL-15Rα).

The engineered host cells can be cultured in conventional nutrient media under appropriate culture conditions, (e.g., temperature, pH, humidity, O₂ concentration, CO₂ concentration) selected based on the host cell. Optionally, agents for amplifying the heterologous nucleic acid, activating promoters, and/or for selecting transformants can be added to the medium. Appropriate culture media and conditions can be selected by those skilled in the art, and are described, for example, in references such as Freshney, Culture of Animal Cells, a Manual of Basic Technique, third edition, John Wiley and Sons, New York, 1994 (ISBN 0-47-155830-X) and the references cited therein. Expression products corresponding to the nucleic acids of the invention can also be produced in non-animal cells such as plants, yeast, fungi, bacteria and the like. Details regarding cell culture can be found in Payne et al., Plant Cell and Tissue Culture in Liquid Systems Wiley Intersciences, New York, N.Y., 1992 (ISBN 0-47-103726-5); Gamborg and Phillips (eds), Plant Cell, Tissue and Organ Culture, Springer-Verlag (Berlin Heidelberg New York; ISBN 0-38-758068-9), 1995; and Atlas and Parks (eds), The Handbook of Microbiological Media CRC Press, Boca Raton, Fla., 1993 (ISBN 0-84-932944-2).

To ensure long-term, high-yield production of recombinant IL-15Rα (and other polypeptides, such as IL-15) protein stable expression systems are typically used. For example, cell lines which stably express an IL-15Rα polypeptide are transfected using expression vectors which contain viral origins of replication or endogenous expression elements and a selectable marker gene, such as an antibiotic resistance gene. Following introduction of the heterologous nucleic acid into a suitable host cell line, and growth of the host cells to an appropriate cell density, the promoter is either constitutively active or is induced by appropriate means (e.g., temperature shift or chemical induction) and cells are cultured for an additional period to permit high level expression of the encoded polypeptide.

The secreted polypeptide product can then be recovered from the culture medium or supernatant. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art.

Expressed IL-15Rα polypeptides (for example, fusion polypeptides including an extracellular IL-15Rα ligand-binding domain) can be recovered and isolated or purified from cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (for example, using a tagging system such as FLAG or His), hydroxylapatite chromatography, and lectin chromatography. If warranted, protein refolding steps can be used to assist in appropriate configuration of the expressed protein. If desired, high performance liquid chromatography (HPLC) can be employed in the final purification steps. A variety of purification methods are well known in the art and are described in, for example, Harris and Angal, Protein Purification Applications: A Practical Approach IRL Press at Oxford, Oxford, 1996 (ISBN 0-19-963048-8); Scopes, Protein Purification: Principles and Practice 3^{rd} Edition Springer Verlag, NY, 1993 (ISBN 0-38-794072-3); Bollag et al., Protein Methods, 2^{nd} Edition Wiley-Liss, NY, 1996 (ISBN 047-111837-0); Walker, The Protein Protocols Handbook, 2^{nd} Edition Humana Press, NJ, 2002 (ISBN 0-89-603941-2); Janson and Ryden, Protein Purification: Principles, High Resolution Methods and Applications, Second Edition Wiley-VCH, NY, 1998 (ISBN 0-47-118626-0); and Walker Protein Protocols on CD-ROM Humana Press, NJ, 1998.

In addition to the IL-15Rα and IL-15 polypeptides explicitly discussed herein, numerous equivalent polypeptides can be used for producing IL-15Rα/ligand complexes. For example, polypeptides that exhibit the biological activity of an IL-15Rα or IL-15 molecule explicitly designated herein (for example, SEQ ID NOs:2, 3 5, 6 and/or 14) but that differ by one more amino acids are equivalents within the context of the IL-15Rα/ligand complexes disclosed herein. Typically, such a polypeptide shares at least 80% amino acid sequence identity over substantially the entire length of a provided IL-15Rα or IL-15 polypeptide. In other embodiments, other substituted polypeptides share at least 85%, at least 90%, at least 95%, at least 98%, or at least 99% amino acid sequence identity with an IL-15Rα or IL-15 polypeptide disclosed herein, over the entire length, substantially the entire length or over the relevant domain thereof, such as the extracellular ligand-binding domain of the IL-15Rα. Exemplary variant polypeptides are described in U.S. Pat. No. 5,552,303 and in Bernard et al., J. Biol. Chem. 279:24313-24322, 2004, which are incorporated herein by reference.

Sequence identity refers to the similarity between two amino acid sequences (or two nucleic acid sequences), and is frequently measured in terms of percentage identity (or similarity); the higher the percentage, the more similar the two sequences are. Polypeptides that retain biological activity of a native IL-15Rα or IL-15 typically possess a relatively high degree of sequence identity when aligned using standard methods, for example, at least 80%, or 85%, or 90% or 95%, or 98%, or 99% identical amino acid residues as compared to a reference IL-15Rα or IL-15 sequence (such as those of SEQ ID NOs:2, 3 5, 6 and/or 14).

Methods of alignment of sequences for comparison are well known, and can readily be utilized to compare IL-15Rα and IL-15 polypeptides. Various programs and alignment algorithms are described in: Smith & Waterman, Adv. Appl. Math 2:482-489, 1981; Needleman & Wunsch, J. Mol. Biol. 48:443-453, 1970; Pearson & Lipman, Proc. Natl. Acad. Sci. USA 85:2444-2448, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al., Computer Appls. Biosci. 8, 155-65, 1992; and Pearson et al., Meth. Mol. Bio. 24:307-311, 1994 and Pearson et al., Meth. Mol. Bio. 25:365-389, 1994. Altschul et al., J. Mol. Biol. 215:403-10, 1990, presents a detailed consideration of sequence alignment methods and similarity/homology calculations.

The NCBI Basic Local Alignment Search Tool (BLAST) (Altschul et al., J. Mol. Biol. 215:403-10, 1990) is available from several sources, including the National Center for Biotechnology Information (NCBI, Bethesda, Md.) and on the Internet, for use in connection with the sequence analysis programs blastp, blastn, blastx, tblastn and tblastx. Each of these sources also provides a description of how to determine sequence identity using this program. Those of skill in the art are familiar with such algorithms and how to use them.

Alternative IL-15Rα and IL-15 polypeptides can be produced by manipulation of the nucleotide sequence encoding such polypeptides using standard procedures. For instance, in one specific, non-limiting, embodiment, site-directed mutagenesis or in another specific, non-limiting, embodiment, PCR, can be used to produce functionally equivalent but non-identical polypeptides. The simplest modifications involve the substitution of one or more amino acids for amino acids having similar biochemical properties. These so-called conservative substitutions are likely to have minimal impact on the activity of the resultant protein. Table 1 provides a summary of conservative amino acid substitutions. In some instances, the polynucleotide sequence is altered by one or more polynucleotide without altering the amino acid sequence of the encoded polypeptide.

TABLE 1
Conservative Amino Acid Substitutions

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

In addition to sequence similarity, a measure of similarity between nucleic acids molecules is the ability to specifically hybridize. Thus, functionally equivalent IL-15Rat and IL-15 polypeptides that are encoded by nucleic acids that specifically hybridize to an IL-15Rα or IL-15 nucleic acid explicitly disclosed herein (such as SEQ ID NOs:1, 4 and/or 13) are suitable for the production of IL-15/IL-15Rα complexes. Specific hybridization refers to the binding, duplexing, or hybridizing of a molecule only or substantially only to a particular nucleotide sequence (such as SEQ ID NO:1, 4 and/or 13) when that sequence is present in a complex mixture (e.g., total cellular DNA or RNA). Specific hybridization can occur under conditions of varying stringency.

Hybridization conditions resulting in particular degrees of stringency will vary depending upon the nature of the hybridization method of choice and the composition and length of the hybridizing DNA used. Generally, the temperature of hybridization and the ionic strength (especially the Na+ concentration) of the hybridization buffer will determine the stringency of hybridization. Calculations regarding hybridization conditions required for attaining particular degrees of stringency are discussed by Sambrook et al., Molecular Cloning—A Laboratory Manual (3rd Ed.), Vol. 1-3, Cold Spring Harbor Laboratory Press, Cold Spring Harbor, N.Y., 2001 (ISBN 0-87-969577-3). By way of illustration only, a hybridization experiment can be performed by hybridization of a DNA molecule to a target DNA molecule which has been electrophoresed in an agarose gel and transferred to a nitrocellulose membrane by Southern blotting (Southern, J. Mol. Biol. 98:503-517, 1975), a technique well known in the art and described in Sambrook et al., Molecular Cloning—A Laboratory Manual (3rd Ed.), Vol. 1-3, Cold Spring Harbor Laboratory Press, Cold Spring Harbor, N.Y., 2001 (ISBN 0-87-969577-3).

Traditional hybridization with a target nucleic acid molecule labeled with [³²P]-dCTP is generally carried out in a solution of high ionic strength such as 6× SSC at a temperature that is 20-25° C. below the melting temperature, Tₘ, described below. For Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal.

The term Tₘ represents the temperature (under defined ionic strength, pH and nucleic acid concentration) at which 50% of the probes complementary to the target sequence hybridize to the target sequence at equilibrium. Because the target sequences are generally present in excess, at Tₘ 50% of the probes are occupied at equilibrium. The Tₘ of such a hybrid molecule can be estimated from the following equation (Bolton and McCarthy, Proc. Natl. Acad. Sci. USA 48:1390-1397, 1962):
Tₘ=81.5° C.−16.6(log₁₀[Na⁺])+0.41(% G+C)−0.63(% formamide)−(600/l)
where l =the length of the hybrid in base pairs.

This equation is valid for concentrations of Na⁺ in the range of 0.01 M to 0.4 M, and it is less accurate for calculations of Tm in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 11 of Sambrook et al., Molecular Cloning—A Laboratory Manual (3rd Ed.), Vol. 1-3, Cold Spring Harbor Laboratory Press, Cold Spring Harbor, N.Y., 2001 (ISBN 0-87-969577-3).

IL-15Rα (and IL-15) nucleic acids and/or polypeptides can be manipulated using well known molecular biology techniques. Detailed protocols for numerous such procedures are described in, for example, Sambrook et al., Molecular Cloning—A Laboratory Manual (3rd Ed.), Vol. 1-3, Cold Spring Harbor Laboratory Press, Cold Spring Harbor, N.Y., 2001 (ISBN 0-87-969577-3) (“Sambrook”); and Ausubel et al., Current Protocols in Molecular Biology (supplemented through 2004) John Wiley & Sons, New York (“Ausubel”).

In addition to the above references, protocols for in vitro amplification techniques, such as the polymerase chain reaction (PCR), the ligase chain reaction (LCR), Qβ-replicase amplification, and other RNA polymerase mediated techniques (e.g., NASBA), useful e.g., for amplifying cDNA probes of the invention, are found in U.S. Pat. No. 4,683,202; and in Innis et al. (eds), PCR Protocols A Guide to Methods and Applications Academic Press Inc. San Diego, Calif., 1990 (0-12-372181-4); Bartlett and Stirling (Eds), PCR Protocols (Methods in Molecular Biology), Humana Press, Totowa, N.J., 2003 (ISBN 0-89-603627-8).

Formation of Complexes

IL-15Rα/ligand complexes are produced by contacting polypeptides (such as fusion polypeptides) including the extracellular ligand-binding domain of IL-15Rα with an appropriate ligand, such as an IL-15 polypeptide. Upon contact, the ligand stably associates with the extracellular portion of the IL-15Rα with high affinity. Typically, the polypeptides are contacted in an aqueous medium, such as a buffered salt solution or cell culture medium. A ligand, such as IL-15 is added to a solution containing the IL-15Rα polypeptide and permitted to associate until equilibrium binding is attained. In the presence of sufficient IL-15, virtually all of the available IL-15Rα ligand-binding domains become associated with IL-15 due to the high binding affinity of the receptor for its ligand. Therefore, it is common to contact a given amount of IL-15Rα polypeptide with at least an equimolar amount of IL-15. More typically, a molar excess of IL-15 is used, such as a 1.5:1, or a 2:1, or a 3:1, or greater molar excess (e.g., 5:1 or even 10:1) of IL-15 to IL-15Rα polypeptide is used to ensure that most, if not all, of the IL-15α polypeptide of the proper conformation is associated with IL-15. Ratios of a selected ligand sufficient to ensure formation of complexes with essentially all of the available IL-15Rα can be determined empirically without undue experimentation by one of skill in the art.

For example, following growth of host cells for a period of time sufficient to allow for accumulation of the desired amount of polypeptide as described above, recombinant polypeptides with the extracellular ligand-binding domain of IL-15Rα are obtained from the cell culture supernatant and contacted with IL-15 polypeptide. Depending on the characteristics of the polypeptide including the extracellular ligand-binding domain of IL-15Rα, additional processing can be accomplished before or after association of IL-15 with the receptor component to produce multi-subunit IL-15Rα/ligand complexes. Optionally, the polypeptide including the extracellular ligand-binding domain of IL-15Rα is isolated or purified (for example, enriched with respect to cell culture supernatant) prior to or after contacting with the ligand.

In an embodiment, recombinant IL-15RαIgFc polypeptides expressed in host cells is secreted into the cell culture supernatant as a dimer joined by disulfide bonds. Thus, complexes can simply be produced by adding ligand to the supernatant where it spontaneously associates with the IL-15RαIgFc dimers.

Optionally, the IL-15Rα/ligand activator complexes are separated from the solution in which association of the ligand and receptor occurred. Such purification removes unbound ligand. For example, the receptor-bound and free ligand can be separated by binding to protein A-agarose, and purity of the resulting complex can be assessed by HPLC.

Expanding Populations of Lymphocytes

IL-15Rα/ligand activator complexes can be used to expand populations of lymphocytes in vitro and in-vivo. Optionally, cells expanded in vitro can be transplanted into a subject. For example, IL-15Rα/ligand activator complexes induce proliferation and survival of specific lymphocyte subsets, including NK cells, CD8MP and CD8NKT cells. Activating IL-15Rα/ligand complexes are approximately 100 times more effective than either IL-15 alone or soluble IL-15Rα/IL-15 receptor-ligand pairs without an activator domain (such as an IgG1 Fc domain) for this purpose. Thus, the activating IL-15Rα/ligand complexes disclosed herein are useful in any application in which a high potency analogue of IL-15 is desirable.

The activating IL-15/IL-15Rα complexes disclosed herein can be used to expand lymphocytes (including NK cells, CD8MP cells and/or CD8NKT cells) populations in vitro (or ex vivo) and in vivo to therapeutically sufficient numbers. In some applications, lymphocytes and/or progenitors thereof are obtained from a subject (such as a human or animal subject). For example, NK cells or T cells, and/or their progenitors can be obtained from a subject into whom it is desired to introduce an expanded population of lymphocytes for therapeutic purposes (that is, autologous lymphocytes or progenitors). Alternatively, the lymphocytes and/or progenitors can be obtained from a subject other than the individual into whom such cells are later transplanted (heterologous lymphocytes or progenitors). When heterologous cell populations are used they are often selected to be histocompatible with the recipient subject. That is, the cells are obtained from a donor with one, or more than one, identical major histocompatibility alleles. Preferably, the donor subject is matched for most or all of the major (and optionally, minor) histocompatibility antigens as well as blood antigens. In certain applications, cell lines (for example, NK cell lines) are employed as the source of lymphocytes or progenitors expanded by contacting them with IL-15Rα/ligand activator complexes.

Lymphocyte or lymphocyte progenitors can be obtained from any tissue source that includes significant populations of lymphocytes and/or their progenitors. For example, various lymphocyte and progenitor populations are generally found in sufficient numbers in a variety of tissues, including peripheral blood (including cord blood), bone marrow, spleen and lymph nodes. For therapeutic applications (e.g., in humans), it is common to obtain lymphocytes and their progenitors from peripheral blood or bone marrow. In other applications (for example, experimental) suitable lymphocyte populations can also be obtained from spleen and/or lymph nodes. However, such sources are generally not preferred in human applications.

For example, lymphocyte and their progenitors can be obtained by sampling peripheral blood and/or bone marrow. The lymphocytes/progenitors can be contacted with activating IL-15Rα/ligand complexes without further isolation or purification steps. That is, lymphocytes/progenitors can be contacted with activating IL-15Rα/ligand complexes in mixed populations of cells. In some cases, the mixed populations of cells including lymphocytes and lymphocyte progenitors are simply suspended (optionally, diluted or concentrated) in a suitable physiological buffer solution. More commonly, the cells are suspended in a suitable growth medium, such as various Eagle's medium formulations, RPMI 1640, F-10 (Ham's) Nutrient mixtures, and the like, supplemented with animal serum (such as fetal bovine serum). Optionally, reduced serum formulations such as OPTI-MEM® medium can be used. In an embodiment, mouse lymphocytes are grown in RPMI 1640 medium, supplemented with 10% fetal bovine serum and 55 μM β-mercaptoethanol. In another embodiment, human cells are grown in X-VIVO 10™ (Cambrex, East Rutherford, N.J.), supplemented with 10% human serum. For applications, in which cells are introduced (or reintroduced) into a human subject, it is often preferable to use serum-free formulations, such as AIM V® serum free medium for lymphocyte culture or MARROWMAX® bone marrow medium. Such medium formulations and supplements are available form commercial sources such as Invitrogen (GIBCO), Carlsbad, Calif. The cultures can be supplemented with amino acids, antibiotics, and/or with cytokines to promote optimal proliferation and survival.

Most commonly, whole blood or bone marrow samples are further processed to obtain populations of cells prior to placing the lymphocytes and/or progenitors into culture medium (or buffer). For example, the blood or bone marrow sample can be processed to enrich or purify or isolate specific defined populations of cells. The terms purify and isolate do not require absolute purity; rather, these are intended as relative terms. Thus, for example, a purified lymphocyte population is one in which the specified cells are more enriched than such cells are in its source tissue. A preparation of substantially pure lymphocytes can be enriched such that the desired cells represent at least 50% of the total cells present in the preparation. In certain embodiments, a substantially pure population of cells represents at least 60%, at least 70%, at least 80%, at least 85%, at least 90%, or at least 95% or more of the total cells in the preparation.

Methods for enriching and isolating lymphocytes are well known in the art, and appropriate methods can be selected based on the desired population. For example, in one approach, the source material is enriched for lymphocytes by removing red blood cells. In its simplest form, removal of red blood cells can involve centrifugation of unclotted whole blood or bone marrow. Based on density red blood cells are separated from lymphocytes and other cells. The lymphocyte rich fractions can then be selectively recovered. Lymphocytes and their progenitors can also be enriched by centrifugation using separation mediums such as standard Lymphocyte Separation Medium (LSM) available from a variety of commercial sources. Alternatively, lymphocytes/progenitors can be enriched using various affinity based procedures. Numerous antibody mediated affinity preparation methods are known in the art such as antibody conjugated magnetic beads. Lymphocyte enrichment can also be performed using commercially available preparations for negatively selecting unwanted cells, such as ROSETTE-SEP® density gradient mediums formulated for the enrichment of whole lymphocytes, T cells or NK cells. For example, cells can be isolated using magnetic beads (e.g., MACS®, Miltenyi Biotec) according to the manufacturer's instructions using antibodies specific for murine and human cell surface markers that bind to CD8 and NK cells, respectively.

The lymphocytes and/or lymphocyte progenitors are suspended in an appropriate medium (for example, cell culture medium) to ensure viability during the duration of the treatment with IL-15Rα/ligand activator complexes. The activating IL-15Rα/ligand complexes are added to the cell suspension in sufficient concentration to promote expansion of the lymphocytes. The concentration sufficient to promote expansion in vitro depends a number of factors, including, for example, the species from which the cells, and from which the complexes are derived, the valency of the complexes, the density of cells in suspension, and the length of time the cells are maintained in contact with the complexes. Thus, a range of concentrations of IL-15Rα/ligand activator complexes can be used to expand populations of lymphocytes, including NK cells, CD8MP cells, and CD8NKT cells in vitro. In general, human cells require a lower concentration of activating IL-15Rα/ligand complex than do certain animal cells, such as mouse cells. For example, the activating IL-15Rα/ligand complex can be added to the cell suspension at a concentration of at least 10 pM (10 picoMoles/l=10×10⁻¹² moles/l). Typically, the smallest dose required to give the desired expansion of lymphocytes is provided to reduce cost. Typical doses in humans range from about 50 pM to about 1 nM. In some cases, human cells are contacted with activating IL-15Rα/ligand complexes at a concentration of least about 100 pM. In some instances, higher concentrations are warranted, and the activating IL-15Rα/ligand complexes are present in the suspension at concentrations of about 0.2, 0.3, 0.5, 0.75 or 1 nM. In some cases even higher concentrations are used. In other animals (for example, mouse) higher concentrations are typically required to achieve the desired level of cellular expansion. For example, it is common to add at least about 0.5 nM, or about 1 nM, or even more to mouse lymphocytes. In come cases, higher concentrations, e.g., 5 nM, 10 nM, 25 nM, or more is used to expand the cells.

The populations of lymphocytes expanded in vitro can then be transplanted (introduced or returned) into a subject to enhance an innate or antigen specific immune response. Typically, the expanded lymphocyte populations are introduced into a human subject via intravenous infusion in a pharmaceutically acceptable carrier (such as a physiological saline solution).

Alternatively, the lymphocyte populations are expanded in vivo with the IL-15Rα/ligand activator complexes. Lymphocyte populations are expanded in vivo, by administering the activating IL-15Rα/ligand complexes directly to the subject. Typically, the complexes are administered in a pharmaceutical composition containing the complexes along with a pharmaceutically acceptable carrier or excipient. A pharmaceutical composition containing the activating IL-15Rα/ligand complexes can be administered by a variety of routes. Most commonly, a solution containing the complexes is systemically injected into the subject. Suitable pharmaceutical formulations and administration routes are described in more detail below.

As discussed above with respect to in vitro methods, the concentration of activating IL-15Rα/ligand complexes required to induce expansion in vivo can vary from species to species, and depending on the particular complex formulation used. For example, IL-15RαIgFc/IL-15 complexes are typically administered to animal subjects at between 0.1 μg and 500 μg per kilogram (μg/kg) body weight, although higher or lower effective doses can be determined empirically by those of skill in the art. Appropriate administration doses for other formulations can be extrapolated based on comparable molarity based on weight and valency (number of subunits per complex). For example, to expand lymphocytes in vivo in a human, at least about 0.1 μg/kg is administered to the subject. For example, at least about 0.5 μg/kg or at least about 0.75 μg/kg, such as about 1 μg/kg of a IL-15Rα IgFc/IL-15 complex can be administered to a human subject. Typically, up to about 10 μg/kg of such a complex can be administered to a human subject, although frequently, the dose does not exceed about 5 μg/kg, such as a dose of about 4 μg/kg of the complex. The dosage required to expand lymphocytes is somewhat higher in many other animals. For example, mice respond optimally to approximately 10 to 100 higher concentrations of a similar (species specific) IL-15RαIgFc/IL-15 complex, and are typically administered between about 0.5 and 25 μg (such as between 1 and 10 μg) per dose. Optionally, multiple doses are provided over a period of days, weeks, or months.

For example, NK cells are important mediators of innate immunity, and play a critical role in the body's defense against pathogens as well as tumors. IL-15 is critical for the survival of NK cells in vivo (Cooper et al., Blood 100:3633-3638, 2002), and animals genetically deficient in either IL-15 (Kennedy et al., J. Exp. Med. 191:771-780, 2000) or IL-15Rα (Lodolce et al., Immunity 9:669-676, 1998) are deficient in NK cell as well as CD8⁺ T cell number and function. However, soluble IL-15 has not proven effective in inducing expansion of NK cells to therapeutically relevant levels. Populations of NK cells can be expanded by contacting NK cells or NK cell progenitors in vitro, ex vivo or in vivo.

NK cells or NK cell progenitors can be obtained from a subject. For example, NK cells and/or their progenitors can be obtained by sampling peripheral blood and/or bone marrow of a subject. Optionally, the NK cells and/or NK cell progenitors are enriched. The cells can then be suspended in an appropriate culture medium, and contacted with the activating IL-15Rα/ligand complexes in vitro. Optionally, expanded populations of NK cells can then be returned to, or introduced into the subject. Alternatively, NK cells and/or their progenitors can be obtained from a allogeneic donor, or from a cell line capable of generating NK cell progeny. Alternatively, NK cells are expanded in vivo by administering the complex to a subject directly.

In another example, CD8MP and CD8NKT cells are expanded using IL-15Rα/ligand activator complexes. CD8⁺ T cells are important effectors of cellular immunity, and are components of the adaptive as well as immune response against a variety of pathogens as well as against tumors. Memory CD8+ T cells (CD8MP) are involved in long term immunological memory and are important for rapidly generating CD8⁺ effector cells specific for a variety of pathogens, including viruses, bacteria and intracellular pathogens. CD8MP cells and CD8NKT cell populations are both IL-15-dependent, and animals deficient for either IL-15 or the IL-15Rα are deficient in these populations of T cells (Kennedy et al., J. Exp. Med. 191:771-780, 2000; Lodolce et al., Immunity 9:669-676, 1998). Thus, these populations exemplify T cell populations that can be expanded using activating IL-15Rα/ligand complexes as disclosed herein.

For example, CD8⁺ T cells and/or T cell progenitors, including hematopoietic stem cells, can be obtained as indicated above by sampling peripheral blood and/or bone marrow. The mixed cell sample, or an enriched subset of the sample is suspended in culture medium and contacted with activating IL-15Rα/ligand complexes in vitro. The expanded cells can be introduced into (or reintroduced into) a subject. Alternatively, as indicated above with respect to NK cells, CD8⁺ T cells, including CD8MP and CD8NKT cells can be expanded in vivo by administering activating IL-15Rα/ligand complexes directly to a subject.

Methods for Enhancing Immune Response

Based on their ability to induce expansion of important subsets of lymphocytes involved in innate and adaptive immune responses, the IL-15Rα/ligand activator complexes disclosed herein are useful for enhancing an immune response in a subject. As indicated above, the methods disclosed herein are useful for expanding any population of lymphocytes that is dependent on IL-15 induced signaling for proliferation, survival, differentiation and/or activation. Accordingly, these activating IL-15Rα/ligand complexes are useful for enhancing any immune response that depends at least in part on an IL-15 dependent lymphocyte. Exemplary immune responses that can be enhanced using the complexes disclosed herein include anti-tumor as well as anti-pathogen immune responses, including therapeutic and prophylactic immune responses.

In certain embodiments, an immune response is enhanced by expanding lymphocyte populations in vivo by administering IL-15Rα/ligand activator complexes directly to a subject. Typically, the activating IL-15Rα/ligand complexes are administered in a pharmaceutical composition. Formulations for such compositions are discussed below.

In other embodiments, an immune response is enhanced by expanding a lymphocyte population in vitro and then administering the expanded population of cells to a subject, so called ex vivo methods. In such methods for enhancing an immune response (for example an anti-tumor or anti-pathogen immune response), lymphocytes and/or lymphocyte progenitors, from the subject, from a donor or from a cell line, are expanded in vitro as indicated above. In applications where cells are to be introduced into a subject, especially a human subject, it is especially important that the reagents, such as buffers, cell culture media, the IL-15Rα/ligand complexes, and any reagents used to enrich or select cells before or after expansion are suitable for human use. Typically, reagents prepared according to current good manufacturing procedure (cGMP) guidelines. Additionally, when cells are to be introduced into human subjects for therapeutic purposes, the cells are manipulated and maintained under conditions that reduce the likelihood of adventitious pathogens (for example, serum free growth conditions).

Methods for introducing expanded populations of lymphocytes are well known in the art. Typically, a suspension of the expanded lymphocytes in a suitable physiologically acceptable buffer or carrier is infused through the subject's vein over a period of one or more hours; The subject is monitored for adverse reactions, including fever, chills, hives, a fall in blood pressure, or shortness of breath. Such uncommon side effects can usually be treated, and the infusion continued until the desired number of cells is delivered.

For example, NK cells expanded with activating IL-15Rα/ligand complexes are highly effective mediators of tumor cell death. Therefore, lymphocytes, including NK cells, expanded with IL-15Rα/ligand activator complexes are useful for the treatment of a variety of tumors, including for example, colon adenocarcenoma, primary lung carcinoma, lung metastasis, leukemia, lymphoma and malignant melanoma.

An anti-tumor immune response can be enhanced in a subject (typically, a subject with one or more tumors) either by expanding a population of lymphocytes (particularly including NK cells) in vitro, as described above. Mixed populations of cells (for example, derived from the subject's own blood or bone marrow) can be expanded in vitro, and then reintroduced into the subject. Optionally, the expanded population of lymphocytes is enriched following expansion to reduce introduction of dead and/or irrelevant cell populations into the subject. In some cases, the subject's blood or bone marrow sample is enriched to increase the proportion of NK cells and/or NK cell progenitors prior to expanding these lymphocytes in vitro. In some cases, substantially pure populations of NK cells are used. Methods for producing substantially pure populations of cells are known in the art, and include negative selection methods for removing irrelevant cells and positive selection methods for further purifying the desired population. In other cases, NK cell lines are expanded using activating IL-15Rα/ligand complexes and introduced into a subject to enhance an anti-tumor immune response. Alternatively, an anti-tumor immune response can be enhanced by administering activating IL-15Rα/ligand complexes to the subject. In the course of such in vivo administration, NK cells as well as various other lymphocyte populations are expanded, leading to an enhanced anti-tumor immune response.

In other embodiments, an immune response against a pathogen, such as a viral pathogen (for example, HIV), is enhanced. Immune responses against other pathogens, including bacterial and fungal pathogens as well as parasites (such as intracellular parasites) can also be enhanced according to the methods disclosed herein. NK and CD8⁺ T cell populations involved in anti-pathogen immune responses are expanded either in vitro or in vivo, as described above. For example, mixed populations of lymphocytes including NK and CD8⁺ T cells (such as CD8MP and CD8NKT cells) obtained by sampling blood or bone marrow of a subject can be contacted with activating IL-15Rα/ligand complexes in vitro, with or without prior enrichment. The expanded population of lymphocytes is then introduced into a subject to enhance an anti-pathogen immune response. If desired, the expanded population of lymphocytes can be enriched prior to administration to the subject to reduce introduction of dead and/or irrelevant cells. These methods can be utilized to treat a subject with a pathogen infection, for example, to augment an impaired immune response or to supplement a normal immune response against a pathogen to increase subject's ability to reduce or eliminate the pathogen, or to prevent subsequent reinfection.

Optionally, the cells are also contacted with an antigen derived from or corresponding to the pathogen. In one specific, non-limiting example, the activating IL-15Rα/ligand complexes (such as, IL-15RαIgFc/IL-15 complexes) described herein are administered along with an agent that promotes the production of a cellular immune response (that is, a cytotoxic T lymphocyte (CTL) response). A number of means for inducing cellular responses, both in vitro and in vivo, are known. Lipids have been identified as agents capable of assisting in priming CTL in vivo against various antigens. For example, as described in U.S. Pat. No. 5,662,907, palmitic acid residues can be attached to the alpha and epsilon amino groups of a lysine residue and then linked (e.g., via one or more linking residues, such as glycine, glycine-glycine, serine, serine-serine, or the like) to an immunogenic peptide. The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor specific CTL when covalently attached to an appropriate peptide (see, Deres et al., Nature 342:561, 1989).

It is also possible to prophylactically enhance an immune response against a pathogen (or a tumor), by administering IL-15Rα/ligand activator complexes in conjunction with a vaccine. The activating IL-15Rα/ligand complexes can be administered in the same composition (pharmaceutical formulation) as the vaccine or in a separate composition. When administered in a separate composition, the complexes can be administered at the same or a different time (either before or after the vaccine). In the context of this disclosure, a vaccine includes any composition that is predicted to induce an antigen-specific immune response. Thus, a vaccine typically includes at least one antigen to which an adaptive immune response is desired. In addition to an antibody response, an adaptive immune response to many pathogens requires a cellular immune response, mediated by CD8⁺ T cells. For example, generation of a protective immune response against many viruses, such as HIV, EBV, HBV, HCV, and Influenza, requires an antigen-specific CD8+ T cell response. Thus, the methods of enhancing an immune response against a vaccine are particularly relevant to enhancing protective immune responses against pathogens (or for example, tumors) that require a substantial cellular immune response.

Accordingly, it is common in the practice of the methods disclosed herein for enhancing an immune response in a subject, to first select a subject in need of, or in whom it is desirable to have, an enhanced immune response. Thus, in methods of enhancing an anti-tumor immune response, a subject with one or more tumors in need of treatment is typically selected. In methods for treating a pathogen infection, a subject with such an infection is typically selected. In methods for enhancing an immune response to a vaccine, a subject in whom an immune response to the vaccine antigen is desired is selected. Such a subject can be a subject who has not previously been exposed to the antigen, or a subject who has previously been exposed to the antigen (for example, infected with a pathogen that expresses the antigen or a homologue thereof).

Pharmaceutical Compositions

IL-15Rα/ligand activator complexes can be administered to a subject to expand populations of lymphocytes in vivo as discussed previously. In such methods, a therapeutically effective amount of an activating IL-15Rα/ligand complex is administered to a subject to prevent, inhibit or to treat a condition, symptom or disease, such as a tumor or a disease resulting from exposure to a pathogenic organism. In such methods, the activating IL-15Rα/ligand complexes are administered by any means known to one of skill in the art (see, Banga, A., “Parenteral Controlled Delivery of Therapeutic Peptides and Proteins,” in Therapeutic Peptides and Proteins, Technomic Publishing Co., Inc., Lancaster, Pa., 1995) such as by intravenous, intramuscular, subcutaneous injection or by oral, nasal or anal administration. Commonly, the IL-15Rα/ligand activator complexes are administered in a formulation including a carrier or excipient. A wide variety of suitable excipients are known in the art, including physiological saline, PBS and the like. Optionally, the formulation includes additional components, such as adjuvants (for example, aluminum hydroxylphophosulfate, alum, diphtheria CRM₁₉₇).

A pharmaceutical composition including an activating IL-15Rα/ligand complex is thus provided. The compositions can be administered for therapeutic treatments. In therapeutic applications, a therapeutically effective amount of the composition is administered to a subject suffering from a disease, such as a disease resulting from a tumor or infection by a pathogenic organism, such as a pathogenic virus. Single or multiple administrations of the activating IL-15Rα/ligand complexes are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

Controlled release parenteral formulations can be made as implants, oily injections, or as particulate systems. For a broad overview of protein delivery systems, see, Banga, Therapeutic Peptides and Proteins: Formulation, Processing, and Delivery Systems, 2^{nd} Edition, CRC Press, Boca Raton, Fla., 2005 (ISBN 0-84-931630-8). Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see, Kreuter, Colloidal Drug Delivery Systems, J. Kreuter, ed., Marcel Dekker, Inc., New York, N.Y., pp. 219-342, 1994 (ISBN 0-82-479214-9); Tice & Tabibi, Treatise on Controlled Drug Delivery, A. Kydonieus, ed., Marcel Dekker, Inc. New York, N.Y., pp. 315-339, 199 (ISBN 0-82-478519-3).

Polymers can be used for ion-controlled release. Various degradable and nondegradable polymeric matrices for use in controlled drug delivery are known in the art (Langer, Accounts Chem. Res. 26:537-542, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al. Pharm. Res. 9:425-934, 1992; and Pec, J. Pharm. Sci. Tech. 81:626-30, 1992). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, CRC Press, Boca Raton, Fla., 1993; ISBN 1-56-676030-5). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No.5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

In some embodiments, the activating IL-15Rα/ligand complex is included in a formulation with one or more of a stabilizing detergent, a micelle-forming agent, and/or an oil. Suitable stabilizing detergents, micelle-forming agents, and oils are detailed in U.S. Pat. No. 5,585,103; U.S. Pat. No. 5,709,860; U.S. Pat. No. 5,270,202; and U.S. Pat. No. 5,695,770, all of which are incorporated by reference. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, ZWITTERGENT™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, for example, Schmolka, J. Am. Oil. Chem. Soc. 54:110-116, 1977; and Hunter et al., J. Immunol 127:1244-1250, 1981, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167-3175, 1984. The agent can be provided in an effective amount, for example between 0.5 and 10%, or in an amount between 1.25 and 5%. The oil should be both biodegradable and biocompatible so that the body can break down the oil over time, and so that no adverse affects, such as granulomas, are evident upon use of the oil.

In one specific, non-limiting example, a pharmaceutical composition for intravenous administration (injection or infusion), includes a sufficient amount of the complex to deliver between about 0.1 μg and 10 μg per kilogram (μg/kg) body weight of a dimeric IL-15RαIgFc/IL-15 complex, although higher or lower effective doses can be determined empirically by those of skill in the art. For example, to expand lymphocytes in vivo in a human, at least about 0.1 μg/kg (such as about 0.5 μg/kg or about 1 μg/kg) of such a complex is administered to the subject. Typically, up to about 10 μg/kg of such a complex can be administered to a human subject, although frequently, the dose does not exceed about 5 μg/kg, such as a dose of about 4 μg/kg of the complex. The dosage required to expand lymphocytes is somewhat higher in many other animals. For example, mice respond optimally to approximately 10 to 100 higher concentrations of a similar (species specific) IL-15RαIgFc/IL-15 activator complex. Optionally, multiple doses are provided over a period of days, weeks, or months.

Actual methods for preparing administrable compositions will be known or apparent to those skilled in the art and are described in more detail in such publications as Remington's Pharmaceuticals Sciences, 19^{th} Ed., Mack Publishing Company, Easton, Pa. (1995) (ISBN 0-912734-04-3).

### EXAMPLES

### Example 1

### In Vitro Expansion of Lymphocytes by IL-15Rα/Ligand Complexes

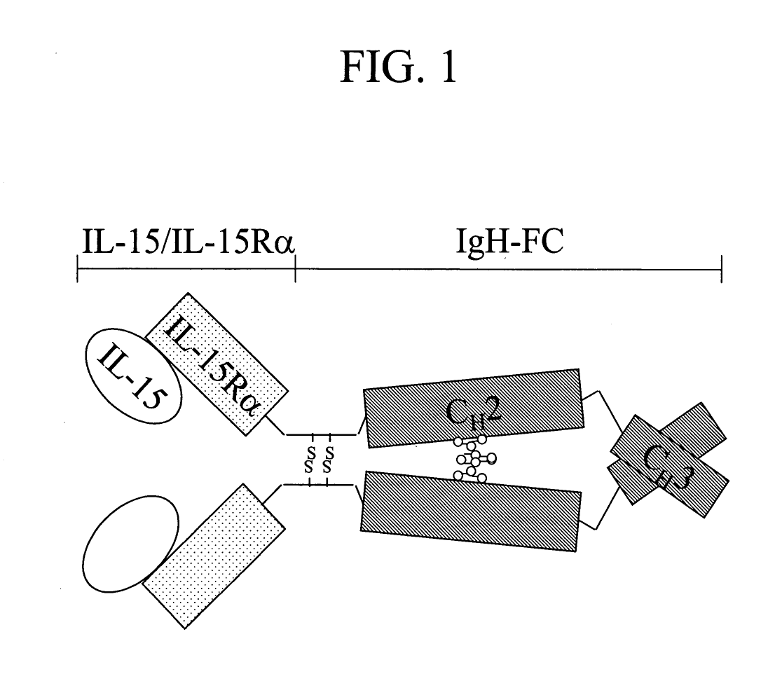

Cells from C57 BL/6 mouse peripheral blood, spleen and bone marrow were cultured in the presence of 1 nM IL-15RαIgFc/IL-15 complex (schematically illustrated in FIG. 1) in RPMI supplemented with 10% fetal bovine serum (FBS) and 55 μM β-mercaptoethanol. Cells were analyzed between 5 and 28 days after the beginning of the cultures. Specific cell subsets were identified by their expression of cell surface markers using flow cytometry.

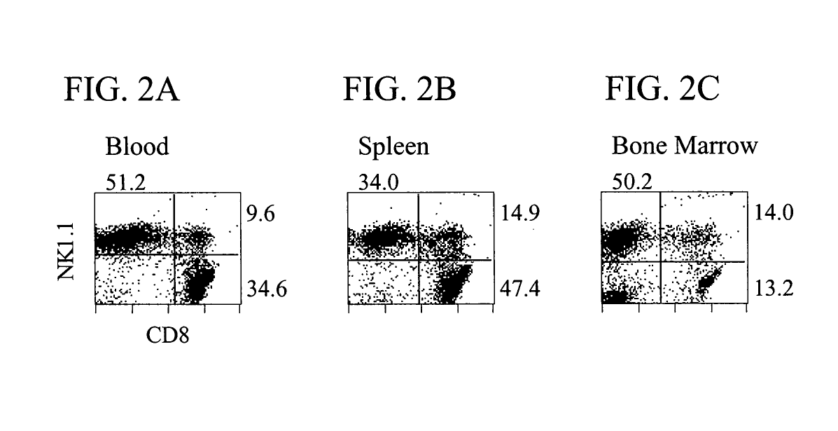

As shown in FIG. 2A, cultures of whole blood with IL-15RαIgFc/IL-15 complexes resulted in an expansion of NK (upper left quadrant), CD8MP (lower right quadrant) and CD8NKT cells (upper right quadrant) resulting in 51.2, 34.6 and 9.6% at day 5, respectively. Similar results were seen if spleen (FIG. 2B) or bone marrow (FIG. 2C) cells were used. Culturing of blood, spleen or bone marrow cells in the absence of IL-15RαIgFc/IL-15 complex leads to the death of all NK, CD8MP and CD8NKT cells after 5 days.

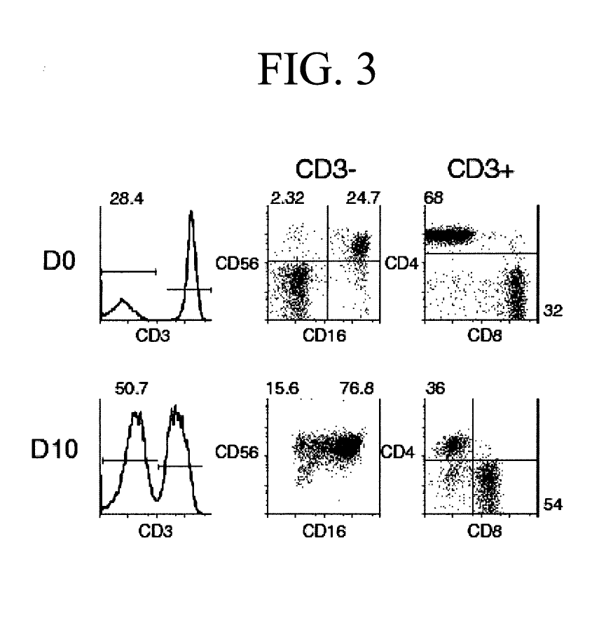

Cells from whole human blood were cultured in X-VIVO 10™, supplemented with 10% human serum. As shown in FIG. 3, cultures of whole human blood resulted in a substantial increase in CD3⁻ cells after 10 days, of which more than 90% were NK cells (CD56⁺). The majority of CD3⁻ cells were CD8-positive after 10 days.

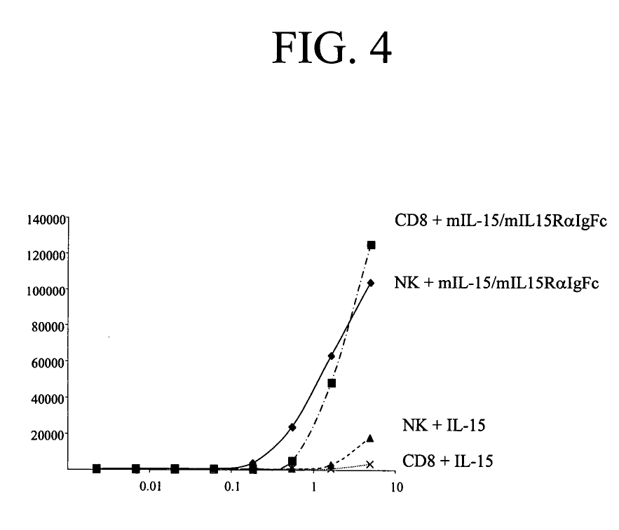

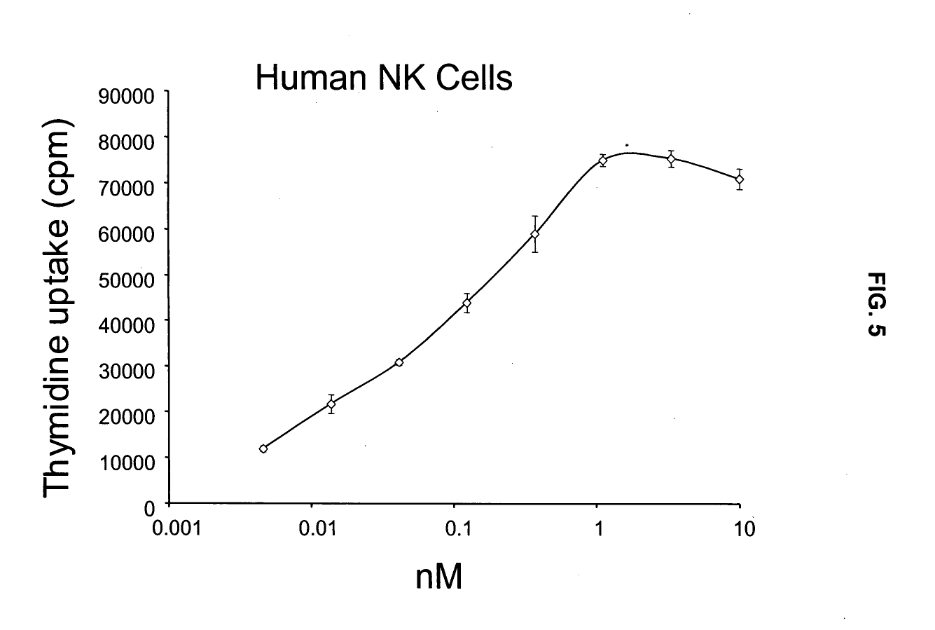

Proliferation in response to IL-15RαIgFc/IL-15 complex was measured by thymidine incorporation following culture with varying concentrations of IL-15RαIgFc/IL-15 complex as shown in FIG. 4. Concentration is indicated on the x-axis in nM/ml, and thymidine incorporation is indicated on the y-axis (cpm). Thymidine was added after 36 hours, and thymidine incorporation was determined after an additional 12 hours of culture. Both NK cells and CD8 cells (CD8MP and CD8NKT) proliferate if IL-15RαIgFc/IL-15 complex is present at a concentration of 1 nM and higher. In contrast, the same concentrations of IL-15 only had only modest effects on the proliferation of these cells. Proliferative response of human NK cells to human IL-15RαIgG1Fc/IL-15 complex is shown in FIG. 5. Increased proliferation of human NK cells is observed at as little as 0.01 nM complex. Proliferation assays were performed as described above.

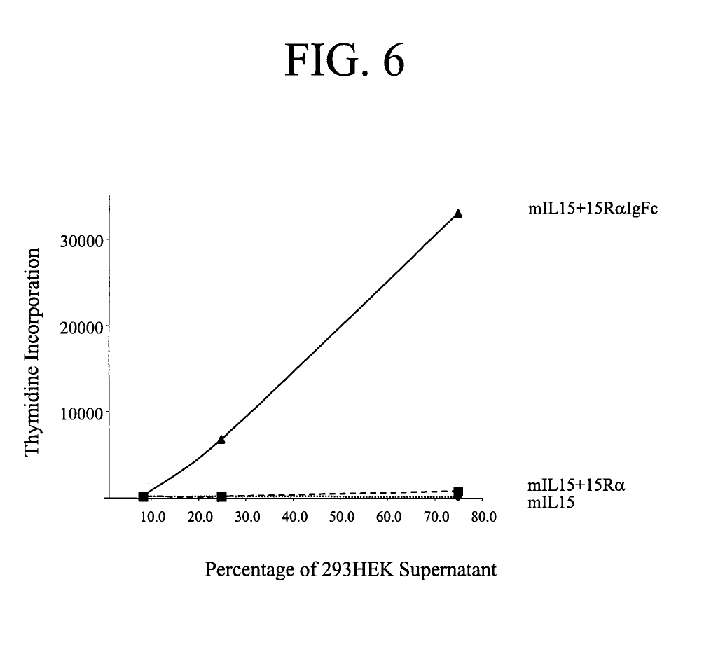

As indicated in FIG. 6, the Fc portion of the IL-15Rα-IgG1Fc/IL-15 complex involved in proliferation. 293HEK cells were co-transfected with nucleic acids encoding the extracellular domain of IL-15Rα and IL-15. Supernatants recovered from 293HEK untransfected and transfected (either with or without the IgFc domain) were added to NK cells. Proliferation was measured as thymidine incorporation (cpm) as described above. The x-axis indicates the percentage of the culture medium that is represented by 293HEK supernatants. IL-15Rα/ligand complex that includes an IL-15RαIgFc fusion polypeptide induces proliferation at supernatant concentrations of 25% and higher. Little proliferation is induced by monomeric IL-15Rα/IL-15 complex lacking the Fc domain, or by IL-15 alone.

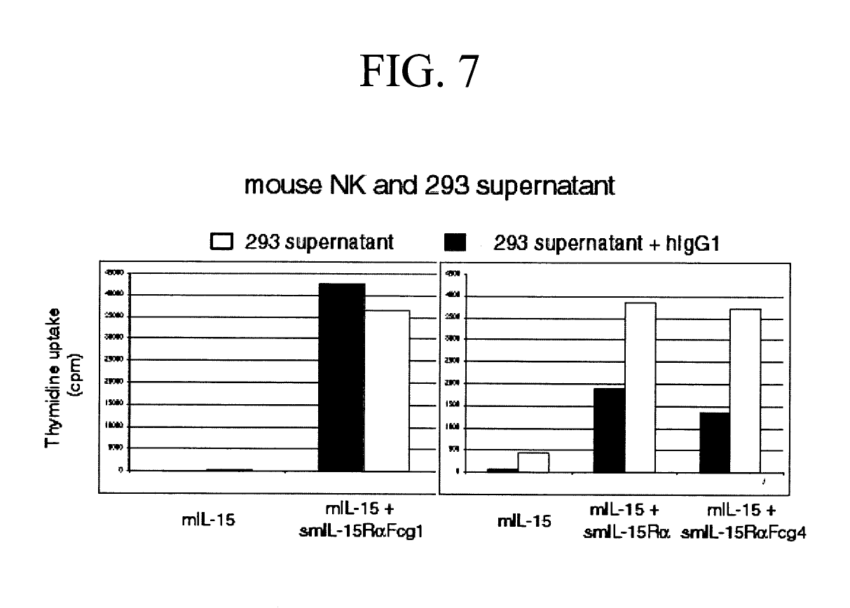

FIG. 7 illustrates proliferation assays of murine NK cells in response to IL-15, IL-15RαIgG1Fc/IL-15, IL-15Rα/IL-15 and IL-15RαIgG4Fc/IL-15, in the presence or absence of cross-linking IgG1. A human monoclonal IgG1 antibody was bound to plates, and the proliferation of NK cells in the presence of various 293HEK-derived supernatants (75% of culture medium) was measured as in FIG. 4. While human monoclonal IgG1 antibody increases the proliferation in response to IL-15, IL-15Rα/IL-15 and IL-15RαIgG4/Fc/IL-15, it decreases the response to IL-15Rα-IgG1Fc/IL-15.

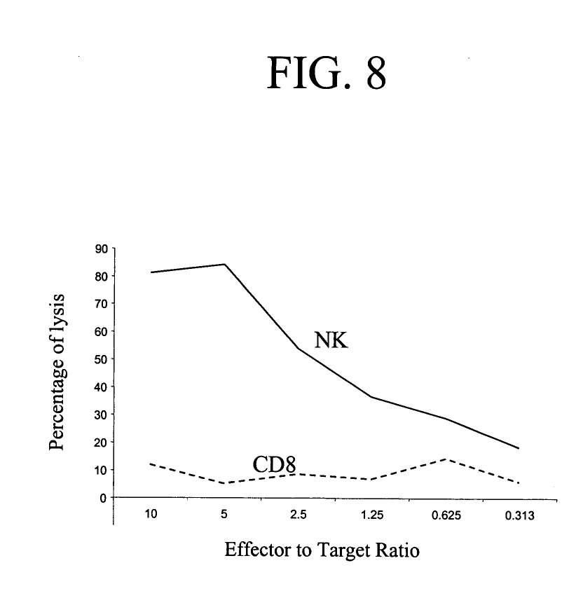

The ability of NK cells and CD8 cells expanded by culture in the presence of ]IL-15RαIgFc/IL-15 complexes to kill syngeneic tumor cells was determined in a chromium release assay. Tumor target cells (2.5×10⁶ MC38 cells) were labeled with 1 mCi chromium-51 in fetal bovine serum for 45 minutes at 37° C. Cells were washed and incubated at different ratios with IL-15RαIgG1Fc/IL-15 expanded lymphocytes. As illustrated in FIG. 8, cultured NK and CD8 cells (both CD8MP and CD8NKT) were co-incubated with a syngeneic tumor cell line (MC38) for 4 hours, and the percentage of lysed tumor cells was measured (y-axis). While NK cells effectively lyse MC38 at NK to tumor cell ratios of 1:1 and higher (x-axis), CD8 cells showed did not exhibit this activity. In a control experiment, NK cells were unable to lyse non-tumor cells.

### Example 2

### Effects of the IL-15RαIgFc/IL-15 Complex In Vivo

To evaluate the effects of activating IL-15Rα/ligand complexes in vivo, between 1 and 10 μg of IL-15RαIgFc/IL-15 complex was injected (i.p.) into C 57 BL/6 mice, and analyzed the percentages of cells in blood and spleen between 3 and 7 days after injection. Specific cells were identified by their expression of specific surface markers using FACS.

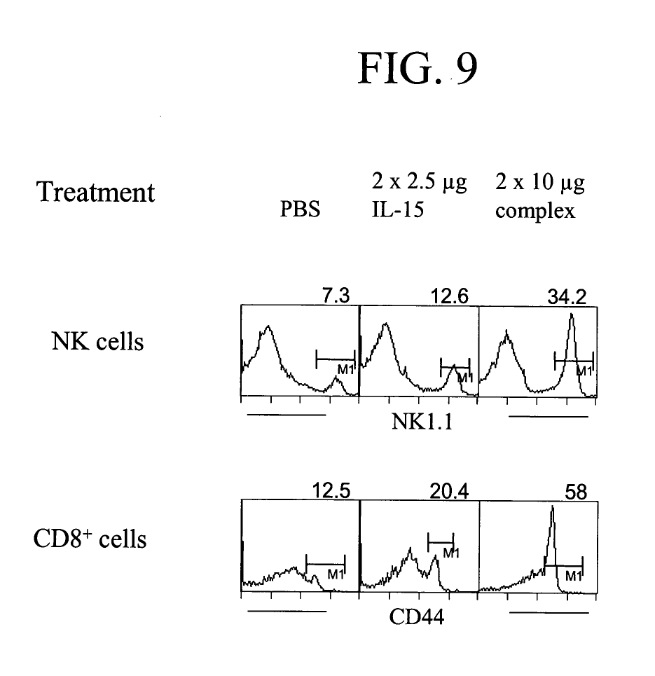

An exemplary analysis is shown in FIG. 9. Molar equivalents of IL-15 (2.5 μg) or IL-15RαIgFc/IL-15 complex (10 μg) were administered by i.p. injection at days 0 and 3. NK cells were identified (upper panel) based on the expression of the cell surface marker NK1.1 (x-axis). Mice injected with phosphate buffered saline (PBS) had approximately 7.3% NK cells in peripheral blood, treatment with 10 μg IL-15RαIgFc/IL-15 complex (twice at days 0 and 3) increased their percentage to 34.2% 7 days after the first injection. Treatment with the equivalent amount of IL-15 had a modest effect on the number of NK cells (12.6%).

The lower panel of FIG. 9 illustrates expansion of CD8MP cells. CD8MP cells were identified by the marker CD44 among the CD8-positive cells (x-axis). Mice injected with PBS contained 12.5% CD8MP cells, whereas the percentage of CD8MP cells was increased to 58% by IL-15RαIgFc/IL-15 complex. As was observed with NK cells in response to IL-15, a modest effect was seen with IL-15 alone.

Additional experiments (BrDU stains) revealed that the expansion of NK, CD8MP and CD8NKT cells after in vivo treatment with IL-15RαIgFc/IL-15 complex is the result of cell proliferation.

FIG. 10 illustrates that a response via the Fc receptor is involved in the expansion of NK cells. Mice were treated with IL-15RαIgG1Fc/IL-15 as shown in FIG. 9. While treatment in this manner led to an expansion of NK cells in wild-type C57 BL/6 mice, no expansion was seen in the blood of mice that were deficient in the FcRγ that is necessary for signaling from Fc receptors.

### Example 3

### Generation of Human IL-15RαIgFc

A sequence encoding a fusion polypeptide including a signal peptide, the mature and extracellular portion of human IL-15Rα and a portion of the constant region of the human IgG1 heavy chain was cloned downstream of a CMV promoter to facilitate expression. The signal peptide from a murine IgG heavy chain (including its own Kozak sequence) was chosen to assure efficient cleavage of the signal peptide. The human IL-15Rα portion encodes amino acids 1-175 of the mature protein (SEQ ID NO:5).

Since the complex has to deliver an activation signal, the 3′ end of the sequence encodes the Fc-portion of the human IgG1 heavy chain. IgG1 was chosen because it is known to activate NK cells. The presence of the Fc-portion enhances the half-life of the fusion polypeptide in vivo, and also facilitates isolation of the protein.

The sequence encoding murine IgG heavy chain Kozak and signal peptide was created by annealing synthetic oligonucleotides (SEQ ID NOs:7 and 8), and inserting the annealed oligonucleotides into a BamH1/EcoR1-digested pCR2.1 plasmid (Invitrogen). Insertion of the annealed oligonucleotides into the vector creates an Eco 47III site at the end of the sequence encoding the signal peptide.

The sequence for the extracellular domain of human IL-15Rα (amino acids 1-175 of the mature protein) was amplified by PCR with oligonucleotide primers corresponding in sequence to SEQ ID NOs:9 and 10 using a cDNA template. The amplification creates an EcoRV site at the 5′ end of the sequence. The 3′ end of the amplified sequence includes 15 bp encoding AA 239-243 of human IgG1 that is followed by an EcoRV site. The EcoRV fragment of this sequence was inserted into the above-described construct, digested with Eco47III and EcoRV.

The sequence for human IgG1 (AA 244-469) including its stop codon was amplified by PCR using human spleen cDNA as template and cloned into pCR2.1 using oligonucleotide primers corresponding in sequence to SEQ ID NOs:11 and 12. To facilitate cloning, the construct contains a degenerate base pair substitutions in the IgG1Fc domain that preserve the amino acid sequence: 1334: C>T. The amplification creates a DraI site at the 5′ end of the product. The DraI/XbaI fragment of this sequence was joined to the extracellular domain of IL-15Rα by ligating the amplified IgG1 domain into the previous construct, which had been digested with EcoRV and XbaI.

The HindIII/XbaI fragment encompassing the entire polynucleotide sequence encoding the IL-15RαIgFc fusion polypeptide was then subcloned into the HindIII/XbaI sites of pCDNA3.1(+) (Invitrogen) under the transcriptional regulatory control of the CMV promoter sequence. The promoter and fusion polypeptide encoding sequence was cloned so that it can be released as a 2078 bp fragment (SEQ ID NO:13) from the vector backbone by digestion with NruI and EcoRI. With respect to SEQ ID NO:13, nucleotides 1-655 encode the CMV Promoter; nucleotides 730-737 encode the Kozak sequence; nucleotides 738-794 encode the murine IgH signal peptide; nucleotides 795-1319 encode the human IL-15Rα extracellular domain; and nucleotides 1320-2022 encode the human IgG1Fc domain.

The resulting NruI/EcoRI fragment was isolated and microinjected into SP2/0 cells (ATCC CRL-1581). SP2/0 cells, which are capable of producing large amounts of recombinant protein. This cell line does not synthesize or secrete any endogenous immunoglobulin chains.

A fusion polypeptide of 425 amino acids is produced (SEQ ID NO:14), in which amino acids 1-19 comprise the murine IgH signal peptide; amino acids 20-194 comprise the human IL-15Rα extracellular domain; and amino acids 195-425 comprise the human IgG1Fc domain.

Supernatant from IL-15Rα-Fc-producing SP2/0 cells is mixed with excess human IL-15 (2-fold based on molarity). Due to its high affinity IL-15 spontaneously binds to IL-15Rα. This mixture separated by binding to protein A-agarose to remove unbound IL-15. Purity of the resulting complex is assessed by HPLC.

### Example 4

### Lysis Activity of sIL-15 Complex-Cultured NK Cells

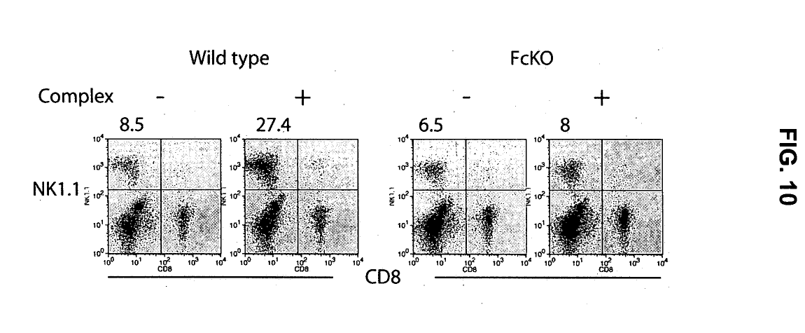

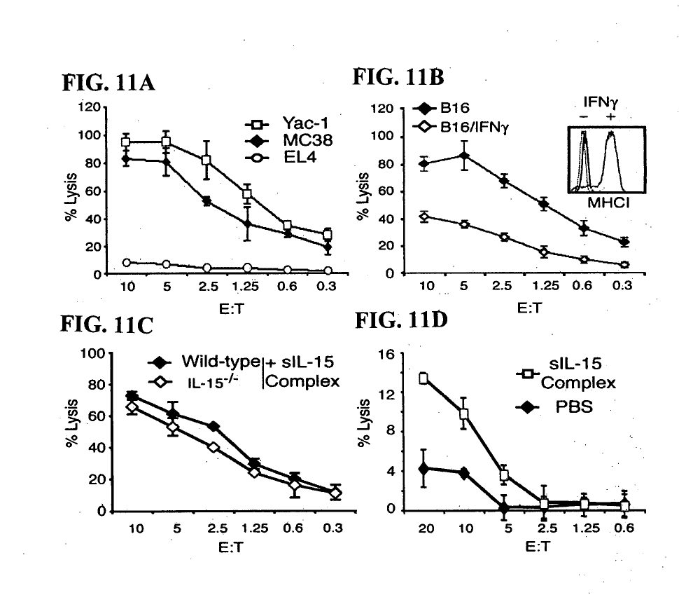

Treatments with IL-15 show some efficiency against tumors in mice (Diab et al., Cytotherapy 7:23-35, 2005). To investigate the feasibility of using sIL-15 complex against tumors it was initially determined whether NK cells cultured with sIL-15 complex retained their cytotoxic activity for tumor cells. NK cells were sorted from spleens and grown for 7 to 14 days in the presence of sIL-15 complex. As lysis target cells we used YAC-1, MC38 and B16. It was previously shown that the effect of IL-15 in inhibiting the pulmonary metastasis following administration of MC38 colon carcinoma cells depended on NK cells (Kobayashi et al., Blood 105:721-72, 2005). FIG. 11A shows that NK cells efficiently lysed both YAC-1 and MC38 but did not target EL4 cells that we used as a non-NK target control. In contrast, little lysis activity was detected in sIL-15-cultured CD8⁺/CD44^{hi} T cells. A preincubation of the NK-sensitive melanoma line B16 with IFN-γ increased the surface expression of MHC I resulting in a loss of NK lysis activity (FIG. 11B, insert shows the induction of MHC I). sIL-15 complex-cultured CD8⁺/CD44^{hi} T cells were unable to lyse B16 regardless of the presence of IFN-γ. The lysis activity of IL-15^{-/-} NK cells was investigated; no significant difference was found when compared with wild-type cells after culturing the cells for 7 days in sIL-15 complex (FIG. 11 C). When freshly isolated NK cells were analyzed, prior injections of sIL-15 complex into mice also increased their cytotoxic activity (FIG. 11D). However, these levels remained well below the cytotoxicity of sIL-15 complex-cultured NK cells. Thus, sIL-15 complex increased the ability of NK cells to lyse target cells.

### Example 5

### Increased Anti-Tumor Effect of sIL-15 Complex

IL-15 has been shown to inhibit tumor growth in various mouse models (Diab et al., supra; Kobayashi et al., supra). It was tested whether pre-associating IL-15 with IL-15Rα-IgG1-Fc would enhance this activity. As a tumor model the melanoma cell line B16 was chosen. This model was efficiently lysed by sIL-15 complex-cultured NK cells (FIG. 11B). When injected intravenously, mice largely develop tumors in the lungs causing death three to four weeks after tumor injection.

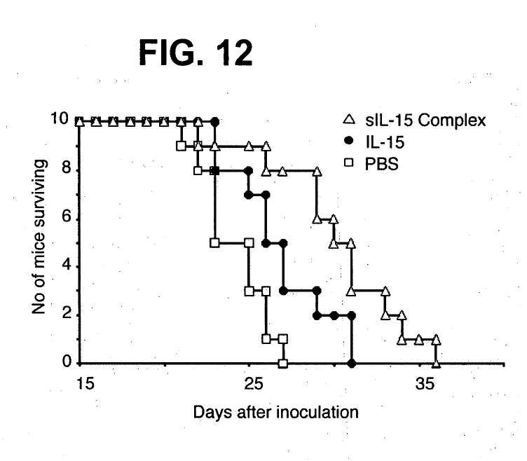

Three groups of ten mice were injected intravenously with 10⁶ B16 cells. Starting three days after tumor injections, mice received nine intraperitoneal injections over three weeks of either PBS, or of approximately equimolar doses of murine IL-15 (2 μg) or murine sIL-15 complex (10 μg). The survival was monitored, and the presence of large pulmonary melanoma masses was confirmed in all mice after death. As shown in FIG. 12, control mice that were mock-treated with phosphate buffered saline (PBS) died with a medium survival of 23 days. Treatments with IL-15 alone increased the survival to 26 days (p=0.0192). The longest medium survival was observed in mice that had been injected with sIL-15 complex (30 days). This survival proved significantly different from both PBS-treated mice (p=0.0003) and from IL-15-treated mice (p=0.0369). Thus, while sIL-15 complex treatment did not cure B16-bearing mice, it proved more efficient in extending the survival than IL-15 alone.

### Example 6

### Additional Results in a Murine Model

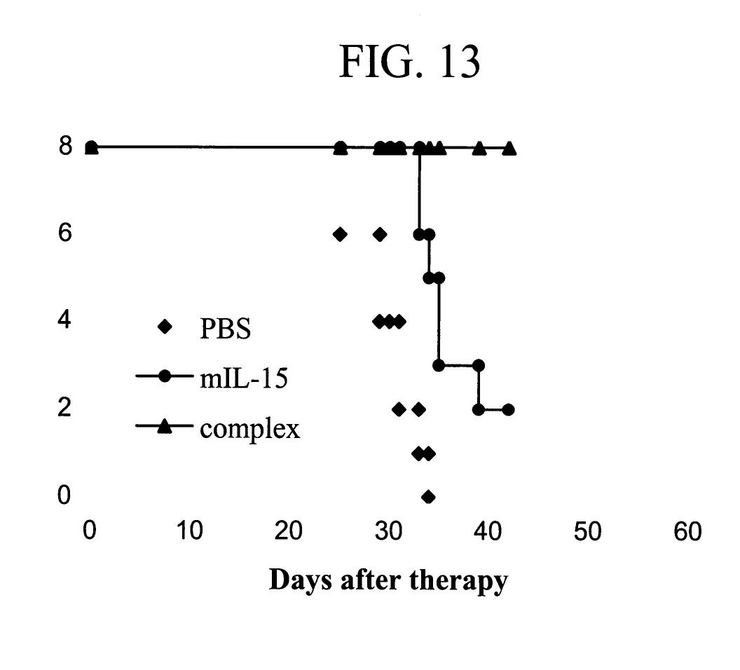

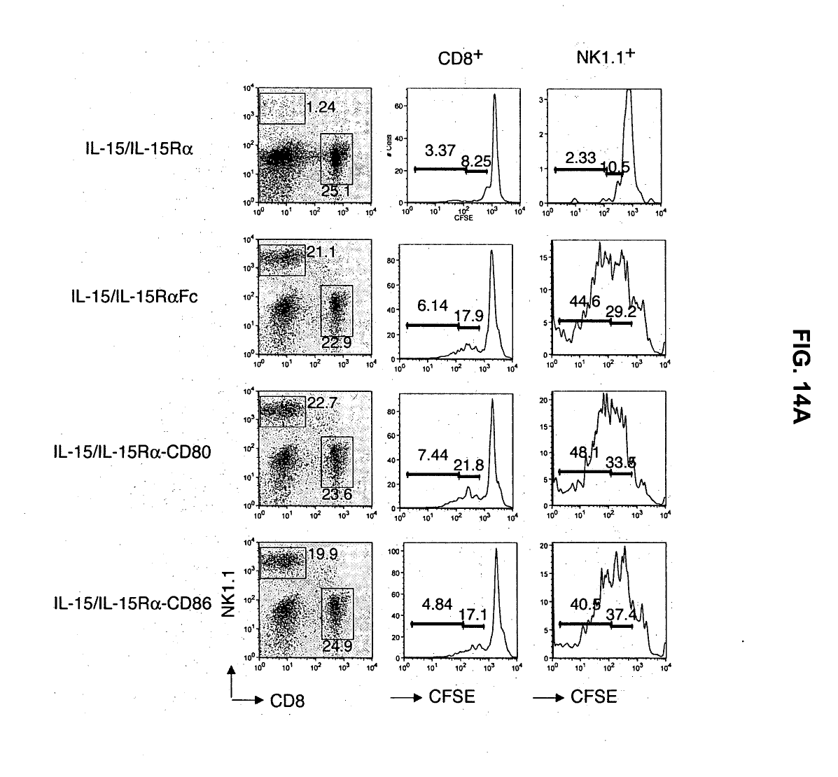

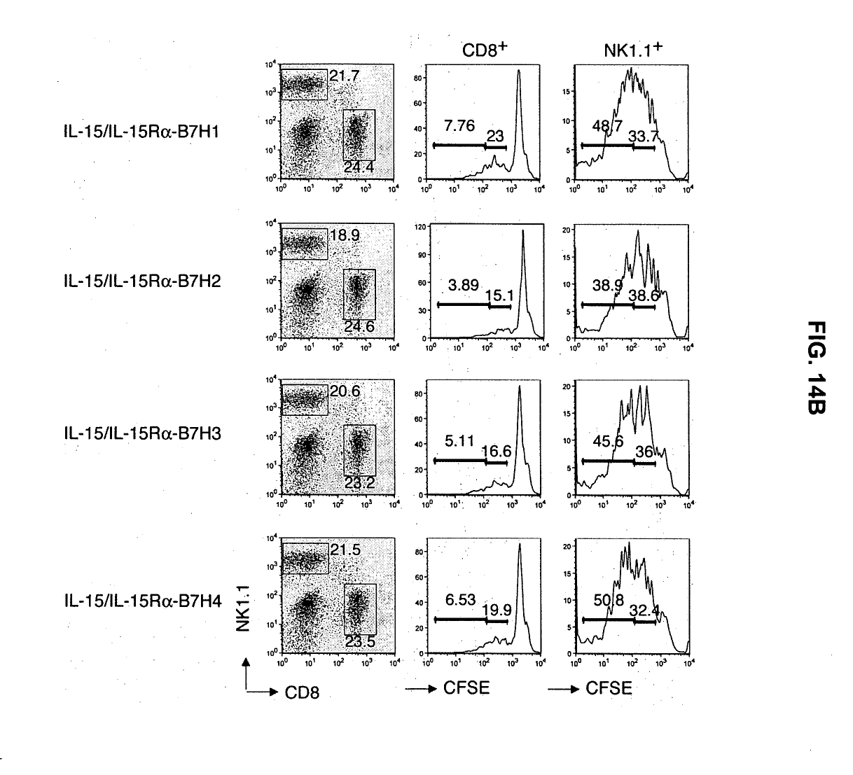

Mice (12 weeks, female) were randomly distributed into three groups of ten animals. One million MC38 tumor cells in 0.2 ml PBS were injected intravenously (i.v.) Treatments were done at day 0 that was followed by three i.p. injections of PBS, 2 μg murine IL-15 or 10 μg murine sIL-15 complex (R&D Systems). The survival of mice was monitored. The presence of melanoma cells in the lungs after death was confirmed for all animals. Statistical significance was determined with the log rank test using GraphPad Prism (GraphPad Software, San Diego, Calif.). sIL-15 complex proved significantly more efficient in inhibiting tumor growth than IL-15 alone (FIG. 13).

### Example 7

### Materials and Methods

Plasmids: Plasmids were constructed using PCR-amplified cDNA fragments from spleen cells and standard cloning techniques. All coding sequences were inserted downstream of a CMV promoter (pcDNA3.1, Invitrogen), a murine Ig Kozak sequence and sequence encoding the murine Ig leader peptide “MAVLVLFLCLVAFPSCVLS” (SEQ ID NO: 15). Sequence encoding the following proteins were cloned downstream of the leader peptide: murine IL-15 aa 30-162; murine IL-15 aa 30-162 followed by human IgG1 aa 239-469 or followed by the human Ig κ-light chain aa 129-236; murine IL-15Rα aa 33-205 followed by an artificial stop codon; murine IL-15Rα aa 33-205 followed by human IgG1 aa 239-469, human IgG2 aa 243-468, human IgG3 aa 292-521 or followed by human IgG4 aa 233-473. A mutation that decreases the binding affinity of human IgG1 to Fc receptors (D265A, (Hakimi et al., J Immunol 151:1075-1085, 1993) was introduced using the QuikChangeII kit (Stratagene). No plasmid encoded unrelated amino acids. Plasmids were expressed in 293HEK cells (ATCC CRL-1573) after transfection with Lipofectamine 2000 (Invitrogen) that resulted in greater than 90% transfection efficiency. Supernatants were collected 48 hours later.

To verify the binding of IL-15 to IL-15Rα, 1 μg of soluble IL-15/IL-15Rα-IgG1-Fc (sIL-15) complex in 1% FBS was immuno-precipitated with protein A/G agarose (Pierce). Proteins from the protein A/G-immuno-depleted supernatant as well as from additional 1 82 g of sIL-15 complex were precipitated in 20% trichloroacetic acid, washed in cold acetone, dried and rehydrated in Laemmli buffer. Both immuno-and protein-precipitated samples were subjected to SDS-PAGE and immuno-blotted with antibodies against IL-15 and IL-15Rα (R&D Systems). To verify the generation of sIL-15 complex by 293HEK cells, 2 ml of supernatants were immuno-precipitated with protein A/G agarose that was followed by SDS-PAGE and immuno-blotting against IL-15 and IL-15Rα.

Mice: C57BL/6 mice were purchased from The Frederick Research Facility, C57BL/6-IL-15^{-/-} and C57BL/6-FcRγ^{-/-} mice were provided by Taconic. All mice used were females between 8 and 12 weeks. All treatments were done by intraperitoneal (i.p.) injection.

Cytometry and Cell Sorting: Antibodies that were used for cytometry were from BD Biosciences. For cytometry analyses, cells were blocked with a mixture of rat IgG1, IgG2a, IgG2b, mouse IgG1 and hamster IgG1 for 15 minutes on ice that was followed by a 30-minute incubation on ice with the specific antibody. For biotinylated antibodies, an additional 15-minute incubation on ice was done with streptavidin-PE-CY5 (BD Biosciences). Bromodeoxyuridine (BrDU) stains were done 12 hours after the i.p. injection of 1 mg BrDU using the BrDU Flow Kit (BD Biosciences). NK cells were sorted from spleens using negative isolation microbeads, and CD8⁺ cells were sorted from spleens using CD8α microbeads (positive sorting) or the CD8⁺ T cell isolation kit (negative sorting, Miltenyi). To determine IgG1-Fc binding, sIL-15 complex was removed from cultured cells and replaced by a high concentration of murine IL-15 (20 nM, Peprotech). Cells were cultured in IL-15 for 12 hours, washed and cytokine-starved for 3 hours. IgG1-Fc binding was detected by incubation with human IL-15Rα-Fc (10 μg/ml, 30 minutes on ice) and staining against IL-15 Rα with a monoclonal antibody (Dubois et al., Immunity 17:537-547, 2002), or by incubation with a biotinylated humanized mouse monoclonal antibody (HuMikBetal (Hakimi et al., J Immunol 151:1075-1085, 1993), 10 μg/ml, 30 minutes on ice) and staining with streptavidin-PE-CY5. Both staining methods gave similar results.

Cell Culture: All cells were cultured in RPMI 1640 supplemented with 10% FBS, 50 μM β-binding mercaptoethanol and antibiotics. Blood cells were cultured after removing erythrocytes via Ficoll-centrifugation. Erythrocytes were removed from spleen cell suspensions by lysis in ACK. Blood and spleen cells were cultured in 1 nM murine sIL-15 complex (provided by R&D Systems). All cytokines were used at concentrations that were indicated by the suppliers. For reasons of simplification, molarities refer to the number of IL-15 molecules even though more than one IL-15 molecule may be part of the protein complexes.

Proliferation Assays: Cells that had been cultured for 7-14 days in 1 nM sIL-15 complex were washed 3 times and plated into 96-well plates at 5*10⁴ per well. Cells were incubated for 48 hours. [³H]Thymidine (1 μCi, Perkin-Elmer) was present during the final 12 hours of the assay. Additional FcR signaling was induced by coating plates with human IgG1 (HuMikBetal, 10 μg/ml in PBS, 4° C., 12 h) before adding cells.

To determine whether cell concentrations affected proliferation in vitro, NK and CD8⁺ T cells were sorted from spleens of untreated mice, stained with CFSE (Molecular Probes, 2.5 μM, 10 min, 37° C.) and cultured in 1 nM sIL-15 complex at various cell concentrations. The dilution of CFSE as a measure of proliferation was determined three days later by FACS.

Lysis Assay: For NK cell-mediated cytotoxicity we used sorted NK cells that had been cultured in 1 nM sIL-15 complex. In addition, the cytotoxic activity of freshly isolated NK cells was determined with or without prior injections of sIL-15 complex (10 μg 7 and 4 days before isolation). As target cells, YAC-1 (ATCC TIB-160), MC38 and B16 were used, as well as EL-4 (ATCC TIB-39). Target cells (2.5*10⁶) were labeled with 1 mCi Chromium-51 (sodium chromate, Amersham) for 1 h at 37° C. in 100% FBS and incubated for 4 hours with effector cells at various effector:target ratios. Supernatants were transferred into 96-well plates (Wallac) and the radioactivity in the liquid phase was measured. Specific lysis was determined by using the formula: % lysis=100*[(mean experimental cpm−mean spontaneous cpm)/(mean maximum cpm−mean spontaneous cpm)). The maximum release value was determined from target cells treated with 1% (v/v) Triton X-100 (Sigma).

B16 tumor protocol: Mice (12 weeks, female) were randomly distributed into three groups of ten animals. One million B16 cells in 0.2 ml PBS were injected intravenously (i.v.) Treatments were done at days 3, 5, 7, 10, 12, 14, 17, 19 and 21 by i.p. injections of PBS, 2 μg murine IL-15 or 10 μg murine sIL-15 complex (R&D Systems). The survival of mice was monitored. The presence of melanoma cells in the lungs after death was confirmed for all animals. Statistical significance was determined with the log rank test using GraphPad Prism (GraphPad Software, San Diego, Calif.).

### Example 8

### Additional Fusions

The amino acids listed below represent the extracellular portions of these activators that were genetically fused to the extracellular portion of IL-15Ralpha. The resulting chimeric proteins were produced together with murine IL-15 in 293HEK cells.

| | | |
|---|---|---|
| CD80 | ACCESSION NM_009855 | amino acids 37-246 of |
| | | SEQ ID NO: 16 |
| Cd86 | ACCESSION NM_019388 | amino acids 25-245 of |
| | | SEQ ID NO: 17 |
| B7-H1 | ACCESSION NM_021893 | amino acids 19-239 of |
| | | SEQ ID NO: 18 |
| B7-H2 | ACCESSION BC029227 | amino acids 47-279 of |
| | | SEQ ID NO: 19 |
| B7-H3 | ACCESSION NM_133983 | amino acids 29-247 of |
| | | SEQ ID NO: 20 |
| B7-H4 | ACCESSION NM_178594 | amino acids 32-261 of |
| | | SEQ ID NO: 21 |

To study the effect of these fusions on proliferation of CD8 and NK cells, murine spleen cells were labeled with CFSE and cultured for 4 days in medium that contained 25% of the 293HEK cell-generated supernatants containing the chimeric IL-15/IL-15Ralpha-activator complexes. At day 4, the dilution of CFSE was determined by FACS as a measure of proliferation. In FIG. 14, proliferation is shown for CD8 cells (FIG. 14, second row) and for NK cells (FIG. 14, third row). Bars indicate the percentage of fast and slow proliferating cells (e.g., 2.13% and 11.7% in upper right panel of FIG. 14). As controls, the proliferation of CD8 and NK cells was determined for IL-15/IL-15Ralpha complex without activator and for IL-15/IL-15Ralpha-IgG1-Fc complex. The data demonstrated that the IgG1-Fc portion can be replaced by other activators within the soluble L-15 complex. These activators result in similar proliferation of CD8 and NK cells.

The IgG1-Fc portion was also replaced by a number of other membrane proteins that have no known lymphocyte activator function:

| | | |
|---|---|---|
| Fcer2a | ACCESSION NM_013517 | amino acids 48-331 of |
| | | SEQ ID NO: 22 |
| Cd209a | ACCESSION NM_133238 | amino acids 76-238 of |
| | | SEQ ID NO: 23 |
| Signr3 | ACCESSION AF373411 | amino acids 76-237 of |
| | | SEQ ID NO: 24 |
| Signr4 | ACCESSION AF373412 | amino acids 41-208 of |
| | | SEQ ID NO: 25 |
| Clec4d | ACCESSION NM_010819 | amino acids 42-219 of |
| | | SEQ ID NO: 26 |
| Clecsf9 | ACCESSION NM_019948 | amino acids 46-214 of |
| | | SEQ ID NO: 27 |
| dectin-2 | ACCESSION AF240357 | amino acids 40-219 of |
| | | SEQ ID NO: 28 |
| Clec4b | ACCESSION NM_027218 | amino acids 40-209 of |
| | | SEQ ID NO: 29 |

When tested in experiments as described above, these proteins did not support the proliferation of CD8 or NK cells.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for expanding a population of lymphocytes, comprising:
contacting one or more lymphocytes, lymphocyte progenitors or both lymphocytes and lymphocyte progenitors, with a complex, wherein the complex comprises (i) a fusion polypeptide comprising an extracellular ligand-binding domain of an interleukin 15 receptor alpha (IL-15Rα) and a lymphocyte-activating domain; and (ii) a ligand of the IL-15Rα, thereby expanding the population of lymphocytes.

2. The method of claim 1, wherein the ligand comprises an IL-15 polypeptide.

3. The method of claim 2, wherein the ligand comprises a variant or fragment of IL-15.

4. The method of claim 1, wherein the one or more lymphocytes, lymphocyte progenitors, or both lymphocytes and lymphocyte progenitors, comprises a natural killer (NK) cell, an NK progenitor cell, or both an NK cell and an NK progenitor cell, thereby expanding a population of NK cells.

5. The method of claim 1, wherein the one or more lymphocytes or lymphocyte progenitors comprises a CD8 expressing lymphocyte, thereby expanding a population of memory T cells.

6. The method of claim 1, wherein the one or more lymphocytes, lymphocyte progenitors or both lymphocytes and lymphocyte progenitors are contacted with the complex in vitro.

7. The method of claim 1, wherein the one or more lymphocytes, lymphocyte progenitors, or both lymphocytes and lymphocyte progenitors are contacted with the soluble complex in vivo.

8. The method of claim 1, wherein the fusion polypeptide comprises a first domain comprising an extracellular IL-15 binding domain and a second domain, which second domain promotes activation of at least one of NK cells, CD8MP cells, CD8NKT cells, and progenitors thereof.

9. The method of claim 8, wherein the second domain comprises an immunoglobulin Fc domain, a CD80 domain, a CD86 domain, a B7-H1domain, a B7-H2 domain, a B7-H3 domain, or a B7-H4 domain.

10. The method of claim 9, wherein the second domain comprises an immunoglobulin Fc domain, and wherein the immunoglobulin Fc domain is an IgG1 Fc domain.

11. The method of claim 6, further comprising administering the expanded population of T cells or NK cells to a subject.

12. The method of claim 11, wherein the subject is a subject with a tumor or a subject with a pathogen infection.

13. The method of claim 12, wherein the pathogen infection is human immunodeficiency virus (HIV).

14. A method for inducing death of a tumor cell, the method comprising:
contacting the tumor cell with at least one of a natural killer (NK) cell and a memory T cell, wherein the NK cell, the memory T cell, or both the NK cell and the memory T cell are a member of a population of cells expanded according to the method of claim 1.

15. A method of treating a subject with cancer, the method comprising:
administering to a subject with cancer a therapeutically effective amount of one or more of:
(a) a soluble complex comprising (i) a fusion polypeptide comprising an extracellular ligand-binding domain of an interleukin 15 receptor alpha (IL-15Rα) and a lymphocyte-activating domain; and (ii) a ligand of the IL-15Rα;
(b) a CD8⁺ memory T cell, wherein the memory T cell is a member of a population of cells expanded ex vivo by contacting at least one memory T cell or progenitor thereof with the soluble complex of (a); and,
(c) a natural killer (NK) cell, wherein the NK cell is a member of a population of cells expanded ex vivo by contacting at least one NK cell or progenitor thereof with the soluble complex of (a).

16. The method of claim 15, wherein the ligand comprises an IL-15 polypeptide.

17. The method of claim 16, wherein the ligand comprises a variant or fragment of IL-15.

18. The method of claim 15, wherein the fusion polypeptide comprises a first domain comprising an extracellular IL-15 binding domain and a second domain, which second domain promotes activation of at least one of NK cells, CD8MP cells, CD8 natural killer (CD8NK)T cells, and progenitors thereof.

19. The method of claim 18, wherein the fusion polypeptide comprises a first domain comprising an extracellular IL-15 binding domain and a second domain comprising an immunoglobulin Fc domain.

20. A method of enhancing an immune response against a pathogen comprising administering to a subject with a pathogen infection one or more of:
(a) a soluble complex comprising (i) a fusion polypeptide comprising an extracellular ligand-binding domain of an interleukin 15 receptor alpha (IL-15Rα) and a lymphocyte-activating domain; and (ii) a ligand of the IL-15Rα;
(b) a CD8⁺ memory T cell, which memory T cell is a member of a population of cells expanded ex vivo by contacting at least one memory T cell or progenitor thereof with the soluble complex of (a); and,
(c) an NK cell, which NK cell is a member of a population of cells expanded ex vivo by contacting at least one NK cell or progenitor thereof with the soluble complex of (a).

21. The method of claim 20, wherein the pathogen is a virus, a bacterium, a fungus or an intracellular parasite.

22. The method of claim 20, wherein the ligand comprises an IL-15 polypeptide.

23. The method of claim 22, wherein the ligand comprises a variant or fragment of IL-15.

24. The method of claim 20, wherein the fusion polypeptide comprises a first domain comprising an extracellular IL-15 binding domain and a second domain, which second domain promotes activation of at least one of NK cells, CD8MP cells, CD8NKT cells, and progenitors thereof

25. The method of claim 24, wherein the fusion polypeptide comprises a first domain comprising an extracellular IL-15 binding domain and a second domain comprising an immunoglobulin Fc domain.

26. A method of enhancing an immune response to a vaccine, the method comprising: administering to a subject:
a therapeutically effective amount of a vaccine composition and a soluble complex comprising (i) a fusion polypeptide comprising an extracellular ligand-binding domain of an interleukin 15 receptor alpha (IL-15Rα) and a lymphocyte-activating domain; and (ii) a ligand of the IL-15Rα.

27. The method of claim 26, wherein the vaccine composition and the soluble complex are administered to the subject simultaneously or sequentially in one or more doses.

28. A pharmaceutical composition comprising a therapeutically effective amount of an activating IL-15Rα/ligand complex and a pharmaceutically acceptable carrier.

29. The pharmaceutical composition of claim 28, wherein the activating IL-15Rα/ligand complex comprises a polypeptide comprising an extracellular ligand-binding domain of an interleukin 15 receptor alpha (IL-15Rα) and a ligand thereof, wherein the activating IL-5Rα/ligand complex comprises lymphoproliferative activity.

30. The pharmaceutical composition of claim 28, wherein the ligand comprises an IL-15 polypeptide.

31. The pharmaceutical composition of claim 30, wherein the ligand comprises a variant or fragment of IL-15.

32. The pharmaceutical composition of claim 28, wherein the polypeptide comprising an extracellular ligand-binding domain of an interleukin 15 receptor alpha (IL-15Rα) is a fusion polypeptide, which fusion polypeptide comprises a first domain comprising an extracellular IL-15 binding domain and a second domain, which second domain promotes activation of lymphocytes.

33. The pharmaceutical composition of claim 32, wherein the fusion polypeptide comprises a first domain comprising an extracellular IL-15 binding domain and a second domain comprising an immunoglobulin Fc domain.

34. The pharmaceutical composition of claim 28, wherein the activating IL-15Rα/ligand complex comprises (a) a fusion polypeptide comprising an extracellular ligand-binding domain of a human interleukin 15 receptor alpha (IL-15Rα) and a human immunoglobulin Fc domain; and (b) a human interleukin 15 polypeptide.
